SEQUENCE LISTING (60 sequences)

SEQ ID NO: 1 - DNA, 38 bp, Artificial
  BMS1084327, forward primer
tgtaaaacgacggccagtttccttccccagacagtcac

SEQ ID NO: 2 - DNA, 27 bp, Artificial
  BMS1084327, reverse primer
gtttctttgtgtatttgggaggggtgt

SEQ ID NO: 3 - DNA, 40 bp, Artificial
  BMS1082942, forward primer
tgtaaaacgacggccagtcatgtgtgtcaacatcaatcca

SEQ ID NO: 4 - DNA, 28 bp, Artificial
  BMS1082942, reverse primer
gtttctttccatccagacaatacagcaa

SEQ ID NO: 5 - DNA, 38 bp, Artificial
  BMS1082956, forward primer
tgtaaaacgacggccagtactggtcaagcagaccatct

SEQ ID NO: 6 - DNA, 27 bp, Artificial
  BMS1082956, reverse primer
gtttcttcccatgttcaggcgttatct

SEQ ID NO: 7 - DNA, 39 bp, Artificial
  BMS1082961, forward primer
tgtaaaacgacggccagtggcaggtgaaaatacttgctg

SEQ ID NO: 8 - DNA, 27 bp, Artificial
  BMS1082961, reverse primer
gtttctttgatgaggcagctcattgac

SEQ ID NO: 9 - DNA, 38 bp, Artificial
  BMS1083945, forward primer
tgtaaaacgacggccagtcaagatgaatgatcccatgc

SEQ ID NO: 10 - DNA, 28 bp, Artificial
  BMS1083945, reverse primer
gtttctttcagcccaggagttaaacatt

SEQ ID NO: 11 - DNA, 38 bp, Artificial
  BMS1083008, forward primer
tgtaaaacgacggccagtgtcctctcagatggcagagc

SEQ ID NO: 12 - DNA, 30 bp, Artificial
  BMS1083008, reverse primer
gtttctttggagacattagtgtgtgctcat

SEQ ID NO: 13 - DNA, 38 bp, Artificial
  BMS1082252, forward primer
tgtaaaacgacggccagtatggtcaccactgcactgac

SEQ ID NO: 14 - DNA, 27 bp, Artificial
  BMS1082252, reverse primer
gtttcttaaggcaggcaagtatttgga

SEQ ID NO: 15 - DNA, 38 bp, Artificial
  BMS1082669, forward primer
tgtaaaacgacggccagtggggagtatgcaattcagga

SEQ ID NO: 16 - DNA, 27 bp, Artificial
  BMS1082669, reverse primer
gtttctttacaggccaaagggaatttg

SEQ ID NO: 17 - DNA, 38 bp, Artificial
  BMS1082702, forward primer
tgtaaaacgacggccagtgcgtgtggatagcgtgagta

SEQ ID NO: 18 - DNA, 27 bp, Artificial
  BMS1082702, reverse primer
gtttcttttgagaccccagtccagaag

SEQ ID NO: 19 - DNA, 38 bp, Artificial
  BMS1082722, forward primer
tgtaaaacgacggccagtggatatcagggagtgggatg

SEQ ID NO: 20 - DNA, 27 bp, Artificial
  BMS1082722, reverse primer
gtttctttcccctgatgttagcagctt

SEQ ID NO: 21 - DNA, 38 bp, Artificial
  BMS1082831, forward primer
tgtaaaacgacggccagtctgctccatatcacgacagc

SEQ ID NO: 22 - DNA, 27 bp, Artificial
  BMS1082831, reverse primer
gtttctttggtcttggtggtctgtttg

SEQ ID NO: 23 - DNA, 38 bp, Artificial
  BMS1887400, forward primer
tgtaaaacgacggccagtgaaaggtggtggtctccttg

SEQ ID NO: 24 - DNA, 27 bp, Artificial
  BMS1887400, reverse primer
gtttctttgagagaagacctggggaga

SEQ ID NO: 25 - DNA, 40 bp, Artificial
  BMS1887404, forward primer
tgtaaaacgacggccagtgggtcgtagagagttgaacaca

SEQ ID NO: 26 - DNA, 30 bp, Artificial
  BMS1887404, reverse primer
gtttcttgctgtctctttcactccaaaatc

SEQ ID NO: 27 - DNA, 38 bp, Artificial
  BMS1784528, forward primer
tgtaaaacgacggccagtctctgagccatatgggaagc

SEQ ID NO: 28 - DNA, 32 bp, Artificial
  BMS1784528, reverse primer
gtttcttttccacagtgtttcagatgtatagc

SEQ ID NO: 29 - DNA, 39 bp, Artificial
  BMS1600436, forward primer
tgtaaaacgacggccagttcaggaagtggtaggcagaga

SEQ ID NO: 30 - DNA, 27 bp, Artificial
  BMS1600436, reverse primer
gtttctttaccactgagccaccagaga

SEQ ID NO: 31 - DNA, 42 bp, Artificial
  CP34, forward primer
tgtaaaacgacggccagtgctgaacaatgtgatatgttcagg

SEQ ID NO: 32 - DNA, 32 bp, Artificial
  CP34, reverse primer
gtttcttgggacaatactgtcttagatgctgc

SEQ ID NO: 33 - DNA, 38 bp, Artificial
  BMS1082043, forward primer
tgtaaaacgacggccagtgggaaacccacacaacagag

SEQ ID NO: 34 - DNA, 65 bp, Artificial
  BMS1082043, reverse primer
gtttcttggagaatggcatggacagagtgtaaaacgacggccagtgggaaacccacacaacagag

SEQ ID NO: 35 - DNA, 39 bp, Artificial
  BMS1082045, forward primer
tgtaaaacgacggccagtttcttcagcactcagccttct

SEQ ID NO: 36 - DNA, 27 bp, Artificial
  BMS1082045, reverse primer
gtttctttcactgctggatatggtgga

SEQ ID NO: 37 - DNA, 40 bp, Artificial
  BMS1081952, forward primer
tgtaaaacgacggccagtttgcaaggttagactttggtga

SEQ ID NO: 38 - DNA, 29 bp, Artificial
  BMS1081952, reverse primer
gtttctttgttcccagaccagtatttcag

SEQ ID NO: 39 - DNA, 38 bp, Artificial
  BMS1081760, forward primer
tgtaaaacgacggccagtctcaaaacgacaaagccaca

SEQ ID NO: 40 - DNA, 27 bp, Artificial
  BMS1081760, reverse primer
gtttcttaggaccggctgtatagcaca

SEQ ID NO: 41 - DNA, 38 bp, Artificial
  BMS1081860, forward primer
tgtaaaacgacggccagtgcaggctggttctttaccac

SEQ ID NO: 42 - DNA, 28 bp, Artificial
  BMS1081860, reverse primer
gtttcttttgtggtaggtttcaccaagg

SEQ ID NO: 43 - DNA, 38 bp, Artificial
  BMS30480882, forward primer
tgtaaaacgacggccagtgcaaatggccaaatgtcatc

SEQ ID NO: 44 - DNA, 27 bp, Artificial
  BMS30480882, reverse primer
gtttcttcatgcaccccaatgttcata

SEQ ID NO: 45 - DNA, 38 bp, Artificial
  BMS30480889, forward primer
tgtaaaacgacggccagttcttgatcactgagccacca

SEQ ID NO: 46 - DNA, 27 bp, Artificial
  BMS30480889, reverse primer
gtttctttcagcaaagaggctggtaca

SEQ ID NO: 47 - DNA, 40 bp, Artificial
  BMS1081770, forward primer
tgtaaaacgacggccagtaaagcgttgctatctgtcacaa

SEQ ID NO: 48 - DNA, 27 bp, Artificial
  BMS1081770, reverse primer
gtttcttgctgtcctgagcacataggg

SEQ ID NO: 49 - DNA, 38 bp, Artificial
  BMS1081774, forward primer
tgtaaaacgacggccagttggaatcccttggacagaac

SEQ ID NO: 50 - DNA, 27 bp, Artificial
  BMS1081774, reverse primer
gtttcttccctgactcctaatgccatc

SEQ ID NO: 51 - DNA, 38 bp, Artificial
  RSAD2_1, forward primer
tgtaaaacgacggccagttagcaaacatgtgggtggtc

SEQ ID NO: 52 - DNA, 27 bp, Artificial
  RSAD2_1, reverse primer
gtttctttttgcagagccgtatttgtg

SEQ ID NO: 53 - DNA, 42 bp, Artificial
  BMS1081640, forward primer
tgtaaaacgacggccagtttttaggtgtacagcagagtgatg

SEQ ID NO: 54 - DNA, 27 bp, Artificial
  BMS1081640, reverse primer
gtttcttggaggcttggtgtgctacag

SEQ ID NO: 55 - DNA, 38 bp, Artificial
  BMS1080704, forward primer
tgtaaaacgacggccagtactcaccctgagtgctccac

SEQ ID NO: 56 - DNA, 27 bp, Artificial
  BMS1080704, reverse primer
gtttcttctccggggtttctcttctct

SEQ ID NO: 57 - DNA, 37 bp, Artificial
  BMS1080870, forward primer
tgtaaaacgacggccagtaatggggcagcaaagagtt

SEQ ID NO: 58 - DNA, 27 bp, Artificial
  BMS1080870, reverse primer
gtttcttctccggggtttctcttctct

SEQ ID NO: 59 - DNA, 18 bp, Artificial
  M13 sequence
tgtaaaacgacggccagt

SEQ ID NO: 60 - DNA, 7 bp, Artificial
  PIGtail sequence
gtttctt

(19) United States
(12) Patent Application Publication
(10) Pub. No.: US 2011/0033849 A1
(43) Pub. Date: Feb. 10, 2011

(54) OVINE IDENTIFICATION METHOD

(75) Inventors: John Colin McEwan, Mosgiel (NZ); Natalie Kathleen Weston, Dunedin (NZ); Gemma Marie Payne, Dunedin (NZ); Nessa Helena O'Sullivan, Dunedin (NZ); Benoit Nel Etienne Elisabeth Auvray, Dunedin (NZ); Kenneth Grant Dodds, East Taieri (NZ)

Correspondence Address: PFIZER INC;Mary J Hosley, 150 EAST 42ND STREET, MS: 150/02/E112, NEW YORK, NY, 10017-5612, US

(73) Assignee: OVITA LIMITED

(21) Appl. No.: 12/668,506

(22) PCT Filed: Jul. 11, 2008
(86) PCT No.: PCT/NZ2008/000173

§ 371 Date: Oct. 18, 2010

(30) Foreign Application Priority Data
Jul. 13, 2007 (NZ) 556506

(51) Int. Cl. C12Q 1/68; G01N 33/50; C07H 21/04; C07H 21/02

(52) U.S. Cl. 435/6; 436/94; 536/23.1; 536/24.33

(57) ABSTRACT

The invention relates to method for identifying an ovine with a genotype indicative of one or more altered performance traits, the method including the step of detecting, in a sample derived from the ovine, the presence of at least one allele of the CP34 simple sequence repeat (SSR) marker, or at least one allele of a marker in linkage disequilibrium (LD) with CP34, wherein the presence of the allele is indicative of the altered performance traits in the ovine.

### FIELD OF THE INVENTION

The present invention relates to a method for identification of ovine with a genotype indicative of one or more altered performance traits.

### BACKGROUND

Marker assisted selection (MAS) is an approach that is often used to identify animals that possess alteration in a particular trait using a genetic marker, or markers, associated with that trait. The alteration in the trait may be desirable and be advantageously selected for, or non-desirable and advantageously selected against, in selective breeding programs. MAS allows breeders to identify and select animals at a young age and is particularly valuable for hard to measure and sex limited traits. The best markers for MAS are the causal mutations, but where these are not available, a haplotype that is in strong linkage disequilibrium with the causal mutation can also be used. Such information can be used to accelerate genetic gain, or reduce trait measurement costs, and thereby has utility in commercial breeding programs.

Often in MAS, a particular marker is used for identification of animals with alteration in a particular trait, and different markers are used for different traits. For example, in sheep, the Inverdale marker is used to identify sheep with altered prolificacy (Galloway et al. 2000) and a GDF8 marker haplotype can be used to identify sheep with a variant causing increased muscling (Johnson et al. 2005).

It would however be beneficial to have available individual markers that could be used to identify animals with alteration in one or multiple performance traits.

It is therefore an object of the invention to provide a method for identifying an ovine with a genotype indicative of one or more altered performance traits, and/or at least to provide the public with a useful choice.

### SUMMARY OF THE INVENTION

In the first aspect the invention provides a method for identifying an ovine with a genotype indicative of at least two altered performance traits, the method including the step of detecting, in a sample derived from the ovine, the presence of at least one allele of the CP34 simple sequence repeat (SSR) marker, or at least one allele of a marker in linkage disequilibrium (LD) with CP34, wherein the presence of the allele is indicative of the altered performance traits in the ovine.

Preferably the performance trait is selected from the group comprising of: weaning weight (WWT), body weight at 8 months (LW8), body weight at 12 months (LW12), carcass weight (CW), adult ewe weight (EWT), eye muscle width (EMW), eye muscle depth (EMD), eye muscle area (EMA), fat depth (FD), carcass fat weight (FAT), carcass lean muscle weight (LEAN), number of lambs born (NLB), lamb fleece weight (LFW), hogget fleece weight (FW12), ewe (adult) fleece weight (EFW), hogget fibre diameter (FDIAM), and resistance to gastrointestinal parasitic nematode infection.

Preferably the performance trait is selected from the group consisting of: weaning weight (WWT), body weight at 8 months (LW8), body weight at 12 months (LW12), carcass weight (CW), adult ewe weight (EWT), eye muscle width (EMW), eye muscle depth (EMD), eye muscle area (EMA), fat depth (FD), carcass fat weight (FAT), carcass lean muscle weight (LEAN), number of lambs born (NLB), lamb fleece weight (LFW), hogget fleece weight (FW12), ewe (adult) fleece weight (EFW), hogget fibre diameter (FDIAM), and resistance to gastrointestinal parasitic nematode infection.

In one embodiment the performance trait is weaning weight (WWT).

Alternatively the performance trait is body weight at 8 months (LW8).

Alternatively the performance trait is body weight at 12 months (LW12).

Alternatively the performance trait is carcass weight (CW).

Alternatively the performance trait is adult ewe weight (EWT).

Alternatively the performance trait is eye muscle width (EMW).

Alternatively the performance trait is eye muscle depth (EMD).

Alternatively the performance trait is eye muscle area (EMA).

Alternatively the performance trait is fat depth (FD).

Alternatively the performance trait is carcass fat weight (FAT).

Alternatively the performance trait is carcass lean muscle weight (LEAN).

Alternatively the performance trait is number of lambs born (NLB).

Alternatively the performance trait is lamb fleece weight (LFW).

Alternatively the performance trait is hogget fleece weight (FW 12).

Alternatively the performance trait is ewe (adult) fleece weight (EFW).

Alternatively the performance trait is hogget fibre diameter (FDIAM).

Alternatively the performance trait is resistance to gastrointestinal parasitic nematode infection.

Preferably the ovine is altered for at least three, more preferably at least four and most preferably at least five performance traits.

In a further aspect the invention provides a method for identifying an ovine with a genotype indicative of at least one altered performance traits selected from the group consisting of: weaning weight (WWT), body weight at 8 months (LW8), body weight at 12 months (LW12), carcass weight (CW), adult ewe weight (EWT), eye muscle width (EMW), eye muscle depth (EMD), eye muscle area (EMA), fat depth (FD), carcass fat weight (FAT), carcass lean muscle weight (LEAN), number of lambs born (NLB), lamb fleece weight (LFW), hogget fleece weight (FW12), ewe (adult) fleece weight (EFW), hogget fibre diameter (FDIAM), and resistance to gastrointestinal parasitic nematode infection, the method including the step of detecting, in a sample derived from the ovine, the presence of at least one allele of the CP34 simple sequence repeat (SSR) marker, or at least one allele of a marker in linkage disequilibrium (LD) with CP34, wherein the presence of the allele is indicative of the altered performance traits in the ovine.

In one embodiment the performance trait is weaning weight (WWT).

Alternatively the performance trait is body weight at 8 months (LW8).

Alternatively the performance trait is body weight at 12 months (LW12).

Alternatively the performance trait is carcass weight (CW).

Alternatively the performance trait is adult ewe weight (EWT).

Alternatively the performance trait is eye muscle width (EMW)

Alternatively the performance trait is eye muscle depth (EMD).

Alternatively the performance trait is eye muscle area (EMA).

Alternatively the performance trait is fat depth (FD).

Alternatively the performance trait is carcass fat weight (FAT).

Alternatively the performance trait is carcass lean muscle weight (LEAN).

Alternatively the performance trait is number of lambs born (NLB).

Alternatively the performance trait is lamb fleece weight (LFW).

Alternatively the performance trait is hogget fleece weight (FW12).

Alternatively the performance trait is ewe (adult) fleece weight (EFW).

Alternatively the performance trait is hogget fibre diameter (FDIAM).

Alternatively the performance trait is resistance to gastrointestinal parasitic nematode infection.

Preferably the ovine is altered for at least two, more preferably at least three, more preferably at least four and most preferably at least five performance traits.

In a further aspect the invention provides a method for identifying an ovine with a genotype indicative of at least one altered performance traits selected from the group consisting of: weaning weight (WWT), body weight at 8 months (LW8), body weight at 12 months (LW12), carcass weight (CW), adult ewe weight (EWT), eye muscle width (EMW), eye muscle depth (EMD), eye muscle area (EMA), fat depth (FD), carcass fat weight (FAT), carcass lean muscle weight (LEAN), number of lambs born (NLB), lamb fleece weight (LFW), hogget fleece weight (FW12), ewe (adult) fleece weight (EFW), and hogget fibre diameter (FDIAM, the method including the step of detecting, in a sample derived from the ovine, the presence of at least one allele of the CP34 simple sequence repeat (SSR) marker, or at least one allele of a marker in linkage disequilibrium (LD) with CP34, wherein the presence of the allele is indicative of the altered performance traits in the ovine.

In one embodiment the performance trait is weaning weight (WWT).

Alternatively the performance trait is body weight at 8 months (LW8).

Alternatively the performance trait is body weight at 12 months (LW12).

Alternatively the performance trait is carcass weight (CW).

Alternatively the performance trait is adult ewe weight (EWT).

Alternatively the performance trait is eye muscle width (EMW)

Alternatively the performance trait is eye muscle depth (EMD).

Alternatively the performance trait is eye muscle area (EMA).

Alternatively the performance trait is fat depth (FD).

Alternatively the performance trait is carcass fat weight (FAT).

Alternatively the performance trait is carcass lean muscle weight (LEAN).

Alternatively the performance trait is number of lambs born (NLB).

Alternatively the performance trait is lamb fleece weight (LFW).

Alternatively the performance trait is hogget fleece weight (FW12).

Alternatively the performance trait is ewe (adult) fleece weight (EFW).

Alternatively the performance trait is hogget fibre diameter (FDIAM).

Preferably the ovine is altered for at least two, more preferably at least three, more preferably at least four and most preferably at least five performance traits.

Resistance to gastrointestinal parasitic nematode infection can be assessed by measuring fecal egg count—summer lamb challenge (FEC1), fecal egg count—autumn lamb challenge (FEC2), and adult fecal egg count (AFEC). Preferably the nematode is of the genus: Haemonchus, Nematodirus, Teladorsagia or Trichostrongylus. Preferably the nematode is of the species Haemonchus contortus, Nematodirus spathiger, Nematodirus filicollis, Teladorsagia circumcincta, Trichostrongylus colubriformis or Trichostrongylus vitrinus.

Preferably the marker in LD with CP34 is an SSR marker.

Preferably the SSR in LD with CP34 is selected from the group including but limited to BMS1084327, BMS1082942, BMS1082956, BMS1082961, BMS1083945, BMS1083008, BMS1082252, BMS1082669, BMS1082702, BMS1082722, BMS1082831, BMS1887400, BMS1887404, BMS1784528, BMS1600436, BMS1082043, BMS1082045, BMS1081952, BMS1081760, BMS1081860, BMS30480882, BMS30480889, BMS1081770, BMS1081774, RSAD2_{—}1, BMS1081640, BMS1080704, and BMS1080870 as herein defined.

More preferably the SSR in LD with CP34 is selected from the group consisting of BMS1084327, BMS1082942, BMS1082956, BMS1082961, BMS1083945, BMS1083008, BMS1082252, BMS1082669, BMS1082702, BMS1082722, BMS1082831, BMS1887400, BMS1887404, BMS1784528, BMS1600436, BMS1082043, BMS1082045, BMS1081952, BMS1081760, BMS1081860, BMS30480882, BMS30480889, BMS1081770, BMS1081774, RSAD2_{—}1, BMS1081640, BMS1080704, and BMS1080870 as herein defined.

In one embodiment the method, the allele of CP34 is selected from a group comprising: allele A, allele B, allele C, allele D, allele E, allele F, allele G and allele H as herein defined.

Preferably the allele of CP34 is allele A, G or H. More preferably the allele of CP34 is allele A. These alleles are particularly suitable to be selected for in sheep breeding programs.

Alternatively the allele of CP34 is allele C or E. Alternatively the allele of CP34 is allele E. These alleles are particularly suitable to be selected against in sheep breeding programs.

Preferably the allele of the marker in LD with CP34, is in LD with CP34 at a D′ value of at least 0.1, more preferably at least 0.2, more preferably at least 0.3, more preferably at least 0.4, more preferably at least 0.5.

Preferably the allele of the marker in LD with CP34, is in LD with CP34 at a V² value of at least 0.05, more preferably at least 0.075, more preferably at least 0.1, more preferably at least 0.2, more preferably at least 0.3, more preferably at least 0.4, more preferably at least 0.5.

Preferably the allele of the marker is in LD with the traits at a D′ value of at least 0.1, more preferably at least 0.2, more preferably at least 0.3, more preferably at least 0.4, more preferably at least 0.5.

Preferably the allele of the marker is in LD with the traits at a V² value of at least 0.05, more preferably at least 0.075, more preferably at least 0.1, more preferably at least 0.2, more preferably at least 0.3, more preferably at least 0.4, more preferably at least 0.5.

The allele may be detected by any suitable method. Preferably the allele is detected using a polymerase chain reaction (PCR) step. PCR methods are well known to those skilled in the art and are described for example in Mullis et al., Eds. 1994 The Polymerase Chain Reaction, Birkhauser, incorporated herein by reference. Preferably a PCR product is produced by amplifying the marker with primers comprising sequence complimentary to sequence of the ovine genome flanking the marker.

Any suitable primer pair may be used. Preferably the PCR is performed using at least one primer selected from those set forth in Table 2. Preferably the PCR is performed using at least one primer pair selected from those set forth in Table 2.

Preferably the allele is identified by the size of the PCR product amplified. Preferably size is estimated by running the PCR product through a gel. Preferably a size standard is also run in the gel for comparison with the PCR product.

Other methods for detecting the allele are also contemplated, such as but not limited to probe-based methods, which are well known to those skilled in the art as described in Sambrook et al., Molecular Cloning: A Laboratory Manual, 2nd Ed. Cold Spring Harbor Press, 1987, incorporated herein by reference.

Beneficially in the method of the invention, the presence of a combination of more than one allele of the CP34 SSR marker, or more than one allele of a marker in linkage disequilibrium (LD) with CP34, may be detected to identify the ovine. Detection of various combinations of alleles of the CP34 SSR and/or alleles of a marker in LD with CP34, commonly known as haplotypes is contemplated.

In a further aspect the invention provides a method for selecting an ovine with at least two altered performance traits, the method comprising selecting an ovine identified by a method of the invention.

In a further aspect the invention provides a method for identifying an ovine with a genotype indicative of at least one altered performance traits selected from the group consisting of: weaning weight (WWT), body weight at 8 months (LW8), body weight at 12 months (LW12), carcass weight (CW), adult ewe weight (EWT), eye muscle width (EMW), eye muscle depth (EMD), eye muscle area (EMA), fat depth (FD), carcass fat weight (FAT), carcass lean muscle weight (LEAN), number of lambs born (NLB), lamb fleece weight (LFW), hogget fleece weight (FW12), ewe (adult) fleece weight (EFW), hogget fibre diameter (FDIAM), and resistance to gastrointestinal parasitic nematode infection, the method comprising selecting an ovine identified by a method of the invention.

In a further aspect the invention provides a method for identifying an ovine with a genotype indicative of at least one altered performance traits selected from the group consisting of: weaning weight (WWT), body weight at 8 months (LW8), body weight at 12 months (LW12), carcass weight (CW), adult ewe weight (EWT), eye muscle width (EMW), eye muscle depth (EMD), eye muscle area (EMA), fat depth (FD), carcass fat weight (FAT), carcass lean muscle weight (LEAN), number of lambs born (NLB), lamb fleece weight (LFW), hogget fleece weight (FW12), ewe (adult) fleece weight (EFW), and hogget fibre diameter (FDIAM), the method comprising selecting an ovine identified by a method of the invention.

In a further aspect the invention provides an isolated polynucleotide comprising an SSR marker selected from the group consisting of BMS1084327, BMS1082942, BMS1082956, BMS1082961, BMS1083945, BMS1083008, BMS1082252, BMS1082669, BMS1082702, BMS1082722, BMS1082831, BMS1887400, BMS1887404, BMS1784528, BMS1600436, BMS1082043, BMS1082045, BMS1081952, BMS1081760, BMS1081860, BMS30480882, BMS30480889, BMS1081770, BMS1081774, RSAD2_{—}1, BMS1081640, BMS1080704, and BMS1080870 as herein defined.

In a further aspect the invention provides a primer suitable for amplifying a polynucleotide of the invention. Preferably the primer comprises sequence complimentary to sequence of the ovine genome flanking the marker. Preferably the primer comprises flanking sequence from the primers set forth in Table 2. Preferably the primer is selected from those set forth in Table 2.

In a further aspect combinations of the alleles of two or more of the above markers, commonly called a haplotype, could be used.

### DETAILED DESCRIPTION OF THE INVENTION

### Definitions

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

The term “polynucleotide(s),” as used herein, means a single or double-stranded deoxyribonucleotide or ribonucleotide polymer of any length but preferably at least 15 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, ribozymes, recombinant polynucleotides, isolated and purified naturally occurring DNA or RNA sequences, synthetic RNA and DNA sequences, nucleic acid probes or primers and fragments.

The term “primer” refers to a short polynucleotide, usually having a free 3′OH group, that is hybridized to a template and used for priming polymerization of a polynucleotide complementary to the target.

The term “probe” refers to a short polynucleotide that is used to detect a polynucleotide sequence, that is complementary to the probe, in a hybridization-based assay.

The abbreviation “SSR” stands for a “simple sequence repeat” and refers to any short sequence, for example, a mono-, di-, tri-, or tetra-nucleotide that is repeated at least once in a particular nucleotide sequence. These sequences are also known in the art as “microsatellites.” A SSR can be represented by the general formula (N1 N2 . . . Ni)n, wherein N represents nucleotides A, T, C or G, i represents the number of the nucleotides in the base repeat, and n represents the number of times the base is repeated in a particular DNA sequence. The base repeat, i.e., N1 N2 . . . Ni, is also referred to herein as an “SSR motif.” For example, (ATC)4, refers to a tri-nucleotide ATC motif that is repeated four times in a particular sequence. In other words, (ATC)4 is a shorthand version of “ATCATCATCATC.”

The term “complement of a SSR motif” refers to a complementary strand of the represented motif. For example, the complement of (ATG) motif is (TAC).

The term “SSR locus” refers to a location on a chromosome of a SSR motif; locus may be occupied by any one of the alleles of the repeated motif “Allele” is one of several alternative forms of the SSR motif occupying a given locus on the chromosome. For example, the (ATC)8 locus refers to the fragment of the chromosome containing this repeat, while (ATC)4 and (ATC)7 repeats represent two different alleles of the (ATC)8 locus. As used herein, the term locus refers to the repeated SSR motif and the flanking 5′ and 3′ non-repeated sequences. SSR loci of the invention are useful as genetic markers, such as for determination of polymorphism.

It will be appreciated by those skilled in the art that an SSR consists of repeats of a certain motif (e.g. ATC), and that different alleles of the SSR locus may have different numbers of repeats [e.g. (ATC)4 or (ATC)7]. Furthermore, the same motif (ATC) may be present, and repeated at a different and unrelated SSR locus. Therefore an SSR locus is defined by the non-repeated sequences flanking the repeated motif Primers complementary to the non-repeated flanking sequences may be used to amplify the repeated region by polymerase chain reaction (PCR). The PCR products may be separated, by methods described herein, to identify individually possessing different alleles of the SSR locus, with different numbers of repeats. Thus the PCR primer sequences (excluding the italicised M13 and PIGtail sequences) in Table 2, and/or sequences complementary to those primer sequences (excluding the italicised M13 and PIGtail sequences), define the SSR markers specified in that table.

“Polymorphism” is a condition in DNA in which the most frequent variant (or allele) has a population frequency which does not exceed 99%.

The term “an SSR in linkage disequilibrium (LD) with CP34” means that the alleles of the SSR are in LD with the CP34 SSR marker.

The term “linkage disequilibrium” or LD as used herein, refers to a derived statistical measure of the strength of the association or co-occurrence of two independent genetic markers. Various statistical methods can be used to summarize linkage disequilibrium (LD) between two markers but in practice only two, termed D′ and V², are widely used.

“Altered” for any particular performance trait means altered relative to an animal of the same breed that does not possess the specified allele.

“Performance trait” means any trait of commercial significance in sheep breeding. Preferred performance traits include weaning weight (WWT), body weight at 8 months (LW8), body weight at 12 months (LW12), carcass weight (CW), adult ewe weight (EWT), eye muscle width (EMW), eye muscle depth (EMD), eye muscle area (EMA), fat depth (FD), carcass fat weight (FAT), carcass lean muscle weight (LEAN), number of lambs born (NLB), lamb fleece weight (LFW), hogget fleece weight (FW12), ewe (adult) fleece weight (EFW), hogget fibre diameter (FDIAM), and resistance to gastrointestinal parasitic nematode infection.

The applicants have identified several novel SSR markers that are in LD with the CP34 marker. The CP34 marker has previously been reported to be weakly associated with the resistance to parasitic nematode resistance. The applicants have now shown that, surprisingly, the CP34 marker, and several markers in LD with CP34, are strongly associated with several other performance traits in ovine, and strongly associated with parasitic nematode resistance. That is the CP34 marker and the markers in LD with CP34, are themselves in LD with these performance traits.

The invention therefore provides a method for identifying an ovine with a genotype indicative of at least one, and preferably two altered performance traits, the method including the step of detecting, in a sample derived from the ovine, the presence of an allele of the CP34 simple sequence repeat (SSR) marker or an allele of a marker in linkage disequilibrium (LD) with CP34, wherein the presence of the allele is indicative of the altered performance traits in the ovine.

Detecting specific polymorphic markers and/or haplotypes can be accomplished by methods known in the art for detecting sequences at polymorphic sites. For example, standard techniques for genotyping for the presence of single nucleotide polymorphisms (SNPs) and/or SSR markers can be used, such as fluorescence-based techniques (Chen, X. et al., Genome Res. 9(5): 492-98 (1999)), utilizing PCR, LCR, Nested PCR and other techniques for nucleic acid amplification. Specific methodologies available for SNP genotyping include, but are not limited to, TaqMan genotyping assays and SNPIex platforms (Applied Biosystems), mass spectrometry (e.g., MassARRAY system from Sequenom), minisequencing methods, real-time PCR, Bio-Plex system (BioRad), CEQ and SNPstream systems (Beckman), Molecular Inversion Probe array technology (e.g., Affymetrix GeneChip), BeadArray Technologies (e.g., Illumina GoldenGate and Infinium assays) and oligonucleotide ligation assay (OLA—Karim et al., 2000, Animal Genetics 31: 396-399). By these or other methods available to the person skilled in the art, one or more alleles of polymorphic markers, including SSRs, SNPs or other types of polymorphic markers, can be identified.

A number of methods are thus available for analysis of polymorphic markers. Assays for detection of markers fall into several categories, including, but not limited to direct sequencing assays, fragment polymorphism assays, hybridization assays, and computer based data analysis. Protocols and commercially available kits or services for performing multiple variations of these assays are available. In some embodiments, assays are performed in combination or in hybrid (e.g., different reagents or technologies from several assays are combined to yield one assay). The following are non-limiting examples of assays are useful in the present invention.

### Direct Sequencing Assays

In some embodiments of the present invention, markers are detected using a direct sequencing technique. In these assays, DNA samples, such as those derived from for example blood, saliva or mouth swab samples, are first isolated from an ovine using any suitable method. In some embodiments, the region of interest is cloned into a suitable vector and amplified by growth in a host cell (e.g., a bacteria). In other embodiments, DNA in the region of interest is amplified using PCR. DNA in the region of interest (e.g., the region containing the marker of interest) is sequenced using any suitable method, including but not limited to manual sequencing using radioactive marker nucleotides, or automated sequencing. The results of the sequencing are displayed using any suitable method. The sequence is examined and the presence or absence of a given polymorphic marker is determined.

### PCR Assay

In some embodiments of the present invention, polymorphisms are detected using a PCR-based assay. In some embodiments, the PCR assay comprises the use of oligonucleotide primers to amplify a fragment containing the polymorphic marker of interest. Such methods are particularly suitable for detection of alleles of SSR markers. The presence of an additional repeats in such an SSR marker, results in the generation of a longer PCR product which can be detected by gel electrophoresis, and compared to the PCR products from individuals without that allele of the SSR marker.

In other embodiments, the PCR assay comprises the use of an oligonucleotide primer that distinguishes (by hybridisation or non-hybridisation) between an allele containing a specific marker, and alternative alleles. Thus in certain embodiments, if PCR results in a product, then the ovine has the marker, and if no PCR product is produced, the ovine does not have the marker.

### Fragment Length Polymorphism Assays

In some embodiments of the present invention, presence of the marker is detected using a fragment length polymorphism assay. In a fragment length polymorphism assay, a unique DNA banding pattern based on cleaving the DNA at a series of positions is generated using an enzyme (e.g., a restriction endonuclease). DNA fragments from a sample containing the marker of interest will have a different banding pattern samples that do not contain the marker.

### RFLP Assay

In some embodiments of the present invention, presence of the marker is detected using a restriction fragment length polymorphism assay (RFLP). The region of interest is first isolated using PCR. The PCR products are then cleaved with restriction enzymes known to give a unique length fragment for a given polymorphic marker. The restriction-enzyme digested PCR products may be separated by agarose gel electrophoresis and visualized by ethidium bromide staining. The length of the fragments is compared to molecular weight standards and fragments generated from test and control samples, to identify test samples containing the marker.

### CFLP Assay

In other embodiments, presence of the polymorphic marker is detected using a CLEAVASE fragment length polymorphism assay (CFLP; Third Wave Technologies, Madison, Wis.; and U.S. Pat. No. 5,888,780).

### Hybridization Assays

In preferred embodiments of the present invention, presence of a marker is detected by hybridization assay. In a hybridization assay, the presence of absence of a given marker sequence is determined based on the ability of the DNA from the sample to hybridize to a complementary DNA molecule (e.g., a oligonucleotide probe). A variety of hybridization assays using a variety of technologies for hybridization and detection are available. A description of a selection of such assays is provided below.

### Direct Detection of Hybridization

In some embodiments, hybridization of a probe to the marker sequence of interest is detected directly by visualizing a bound probe (e.g., a Northern or Southern assay; See e.g., Ausabel et al. (eds.), Current Protocols in Molecular Biology, John Wiley &amp; Sons, NY, 1991). In these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., agarose gel electrophoresis) and transferred to a membrane. A labeled (e.g., by incorporating a radionucleotide) probe or probes specific for the marker sequence being detected is allowed to contact the membrane under a condition of low, medium, or high stringency conditions. Unbound probe is removed and the presence of binding is detected by visualizing the labeled probe.

### Detection of Hybridization Using “DNA Chip” Assays

In some embodiments of the present invention, the presence of the marker is detected using a DNA chip hybridization assay. In this assay, a series of oligonucleotide probes are affixed to a solid support. The oligonucleotide probes are designed to be unique to a given polymorphic maker sequence. The DNA sample of interest is contacted with the DNA “chip” and hybridization is detected.

In some embodiments, the DNA chip assay is a GeneChip (Affymetrix, Santa Clara, Calif.; See e.g., U.S. Pat. No. 6,045,996) assay. In other embodiments, a DNA microchip containing electronically captured probes (Nanogen, San Diego, Calif.) is utilized (See for example U.S. Pat. No. 6,068,818).

In still further embodiments, an array technology based upon the segregation of fluids on a flat surface (chip) by differences in surface tension (ProtoGene, Palo Alto, Calif.) is utilized (See for example U.S. Pat. No. 6,001,311).

In yet other embodiments, a “bead array” is used for the detection of polymorphic marker (Illumina, San Diego, Calif.; See for example PCT Publications WO 99/67641 and WO 00/39587, each of which is herein incorporated by reference).

### Enzymatic Detection of Hybridization

In some embodiments of the present invention, genomic profiles are generated using a assay that detects hybridization by enzymatic cleavage of specific structures (INVADER assay, Third Wave Technologies; See e.g., U.S. Pat. No. 6,001,567). The INVADER assay detects specific DNA and RNA sequences by using structure-specific enzymes to cleave a complex formed by the hybridization of overlapping oligonucleotide probes.

In some embodiments, hybridization of a bound probe is detected using a TaqMan assay (PE Biosystems, Foster City, Calif.; See e.g., U.S. Pat. No. 5,962,233). The assay is performed during a PCR reaction. The TaqMan assay exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, presence of the marker sequence is detected using the SNP-IT primer extension assay (Orchid Biosciences, Princeton, N.J.; See e.g., U.S. Pat. No. 5,952,174).

### Mass Spectroscopy Assay

In some embodiments, a MassARRAY system (Sequenom, San Diego, Calif.) is used to detect presence of the polymorphic marker (See e.g., U.S. Pat. No. 6,043,031.

### Protein Based Marker Detection

It will be appreciated that if the marker linked to CP34 is in a protein coding region, presence of the marker may result in an amino acid change in the encoded protein. In such cases, any suitable method for detecting the presence of the characteristic amino acid in a protein or polypeptide may be applied. Typical methods involve the use of antibodies for detection of the protein polymorphism. Methods for producing and using antibodies are well known to those skilled in the art and are described for example in Antibodies, A Laboratory Manual, Harlow A Lane, Eds, Cold Spring Harbour Laboratory, 1998.

The polynucleotides, markers, primers and probes of the invention can be used to derive estimates for the association of each allele of the markers, in a reference population measured, for the traits of interest using a variety of statistical methods such as mixed models. These estimates coupled with a derived economic value for each trait can be used to rank individuals based solely on their genotype at a young age, or a mixture of their genotype estimates and selected subsets of the traits of interest. This approach is useful to rank individuals for their breeding worth.

Alternatively, the genotype information that can be generated using the polynucleotides, markers, primers and probes of the invention, may be considered as a fixed or random effect in an animal model Best Linear Unbiased Prediction (BLUP) or via mixed models (Mrode, 1996) where animals have parentage and various combinations of traits recorded. This approach would be useful for young animals that have not been recorded for the traits of primary interest, to rank individuals on their likely future performance.

The above approaches are not limited to detecting only CP34, or markers in LD with CP34, but also to situations where CP34, or markers in LD with CP34, which are included as part of a larger marker set from several additional markers to many thousands of markers, and the combined estimates of all markers are used to estimate the genetic worth of an individual or its likely individual performance.

### BRIEF DESCRIPTION OF THE DRAWINGS

FIG. 1 shows a linkage disequilibrium plot for 847 animals consisting of Coopworth, Perendale Romney, Texel and Composite sires used in the analysis. The upper diagonal lists the pairwise D′ (Dp) LD measurement between markers and the lower diagonal lists the Cramer's V squared (V̂²) LD measurement. The markers are listed in bovine genome order which is the inverse of the ovine genome order. In the current context markers with D′ values with CP34 greater than 0.3 or V² greater than 0.1 are considered to define the boundaries of useful LD. This includes the region defined by BMS1887400 to BMS1081770.

FIG. 2 shows a pairwise plot of the statistical significance of the linkage disequilibrium plot for the 847 animals consisting of Coopworth, Perendale Romney, Texel and Composite sires used in the analysis expressed on a −log 10(p) scale. All markers flanking CP34 and extending to BMS1887400 and BMS1081770 showed very highly significant association via linkage disequilibrium with CP34 with −log 10(p) values ranging from 3.6 to 338.8.

FIG. 3 shows allele effect estimates by marker and the significance of the estimates expressed as probability (p) values.

### EXAMPLES

The invention will now be illustrated with reference to the following non-limiting examples.

### Example 1

### Mapping Performance Traits in Sheep in the Ovine Chromosome 3p Region

### Introduction

The only marker that had been previously described in the ovine chromosome 3p region in this work is CP34 (Ede et al. 1995). Beh et al., (2002) reported a small QTL present in one sire for resistance to parasitic nematode infection as assessed by fecal egg count (FEC) in the autumn at the 5% chromosome wide significance threshold located near CP34. Crawford et al., (2006) reported a similar result for 1 sire for FEC1, and another sire for abomasal adult Ostertagia numbers, but in contrast Davies et al., (2006) found no evidence of segregation in this region for FEC or antibody traits. To the applicant's knowledge no other trait associations have been reported in sheep in this region. In addition, work reported to date has been by linkage mapping which, at the marker density and animal numbers used, only defines a general region of perhaps 40 cM (˜40 Mbp) in length in which the QTL may reside, and has little or no predictive value in industry because no marker allele associations have been determined that can be used on independent groups of animals. In order to create such associations typically marker density has to be higher than several markers per cM and numerous pedigrees need to be tested.

### Methods

### WormFEC Sire Resource

The WormFEC resource (McEwan et al 2006) consists of 987 primarily male progeny tested sheep sourced from New Zealand recorded flocks and consisting of individuals of Coopworth, Romney, Perendale, Texel, Composite and other minor breeds. A subset of 847 sires, derived from 111 flocks, were used for this analysis. There were 126,004 progeny weaned from these sires with a median progeny group size of 117 (range 1-2017).

They consisted of:
Coopworth. The breed sample consisted of 362 industry animals used between 2000 and 2006. All animals in this dataset are more than 50% Coopworth.
Perendale. The breed sample consisted of 148 industry animals used between 2000 and 2006. All animals in this resource are more than 50% Perendale.
Romney. The breed sample consisted of 279 animals used between 2000 and 2006. All animals in this dataset are more than 50% Romney.
Texel. The breed samples consist of 27 animals used between 2000 and 2006. All animals in this dataset are more than 50% Texel.
Composites. There were 31 Composites breed animals from the WormFec Sire resource used 2000-2006.

### Parasite Selection Line Resources

The Romney host resistance parasite selection line was initially created in 1979 and has been recently described by Morris et al (2000). Over the selection period these animals have diverged in fecal egg count (FEC) after a standard challenge by 40 fold. They currently consist of 3 FEC selection lines a low, a high and a control line. For the current work DNA samples were collected from 50 susceptible (high) line, 50 resistant (low) line and 53 control line animals in 1997 and were genotyped for the markers described below.

The Perendale host resistance parasite selection line was initially created in 1985 and has recently been described by Morris et al (2005). Over the selection period these animals have diverged in FEC after a grazing challenge by 4.9 fold. They currently consist of 2 selection lines a low and high FEC line respectively. For the current work DNA samples were collected from 107 susceptible line animals and 128 resistant line animals in 1998 and were genotyped for the markers described below.

### SIL ACE EBVs

Performance recording and estimated breeding values (EBVs) are produced in New Zealand by Sheep Improvement Limited (SIL) a trading entity of Meat & Wool New Zealand. The underlying methodology and system used has been described in a number of papers (Geenty, 2000; Amer, 2000; Newman et al., 2000).

The traits recorded and described in this study and further background to the SIL system is at http://www.sil.co.nz/Technical%20Bullentins/Technical%20Notes/ and trait descriptions from this site are attached to this document including: Young and Walker, (2007a) describing trait measurements and breeding values, Young and Walker (2007b) describing SIL indices and sub indices and economic weights, McEwan (2006) and Young (2006) describing recording for host resistance selection in sheep, and Young (2005) describing New Zealand wide across flock and breed genetic evaluations to produce SIL Advanced Central Evaluation (ACE) EBVs and indices. In turn the SIL ACE EBVs are underpinned by the nationwide central progeny test (CPT) project recently described by McLean et al. (2006).

In brief the genetic evaluations use an across flock and breed multi-trait animal model BLUP analysis for all traits, except for NLB and host resistance traits which used across flock and breed multi-trait repeated measures animal model BLUP. Typically these analyses are done in goal trait group combinations. The outputs of these evaluations for individuals are breeding values corrected for flock, year and breed effects. These breeding values are “shrunken” based on the accuracy of the EBV, primarily in this case being affected by the number of measured progeny. This effect is particularly pronounced for situations where only low progeny numbers have been measured.

This analysis used the June 2007 across flock and breed SIL ACE evaluation EBVs for the sires. Values for the individual sires were downloaded from a direct database query as these estimates are not publicly available except to authorized personnel. Details of the overall evaluation description (Newman, 2007) are attached as an appendix. Only the subset of animals that were genetically linked, as described by the SIL ACE criteria were included in this work.

### SSR Discovery and Mapping Process

Novel ovine SSR markers were identified and validated by the following process. The region of interest from the orthologous section of the bovine genome was processed for suitable dinucleotide SSRs with more than 9 repeats using the program Sputnik and then primers designed using Primer 3 using an independent, but analogous approach to that described by Robinson et al. (2004). The bovine genome assembly used was version 3.1 and is available as ftp://ftp.hgsc.bcm.tmc.edu/pub/data/Btaurus/fasta/ as the Btau20040927-freeze and distances are reported on that basis. The primers had a M13 antisense and PIGtail sequence added to them and were then used to PCR amplify DNA samples in conjunction with a fluorescent M13 oligo as described by Boutin-Ganache et al. (2001) and Saito et al. (2005). The size of the resulting products were then measured using standard manufacturer procedures and protocols on a ABI 3730 sequencer. The primers were first screened over a panel of cattle, sheep and deer samples. Markers that passed the initial screen (i.e. were polymorphic in sheep and of reasonable quality see tables 1 and 2 for a list of primers and markers finally selected) were subsequently genotyped across the International Mapping Flock (IMF; Maddox et al. 2001). This allowed the markers to be mapped confirming their location to the region of interest and their suitability for genotyping. Distances and order reported here used markers available in the latest publicly available map v4.7 (http://rubens.its.unimelb.edu.au/˜jillm/jill.htm) and the genotypes obtained from the present study. CRI-Map was used to do the linkage mapping using the process described by Maddox et al. (2001). Markers that mapped to the appropriate location were then genotyped by the same method for the WormFEC sires and Parasite Selection Line (PSL) resources. Results from all the genotyping from the 3730 were measured as raw allele lengths using ABI GeneMapper 4.0 and reported as fragment lengths in base pair units relative to internal standards. These results were binned into alleles on the basis of a cluster analysis based on Ward's distance using SAS (http://www.sas.com/) to define mean allele lengths and reporting variability. The allele names and bins for each marker are shown in Table 3.

Because of uncertainty of marker order and the quality of the bovine assembly a number of markers were mapped using an ovine 5000 RAD ovine Radiation hybrid panel (Eng et al. 2004). This can more accurately position closely spaced markers and acts as a check on the bovine genome assembly. Panel cell lines were genotyped in duplicate using the primers described above for the RH panel and visualised by running on 2% Agarose gels as described by Band et al. (2000). Presence or absence of markers in each cell line was scored and the resulting data mapped using RHmapper (Slonim et al. 1997).

### Estimation of Linkage Disequilibrium

Linkage disequilibrium measures were calculated using the program LDMAX part of the GOLD package (Abecasis and Cookson, 2000) and the R statistical package (http://www.r-project.org/). The measures D′, the square of Cramer's V and the significance expressed as a probability of the association were calculated for all combinations of markers and plotted using a combination of the graphics facilities in SAS (http://www.sas.com/) and R. Each measure is useful for certain purposes and in this case we used a threshold of D′>0.3 or V²>0.1 and p<1×E-10 with CP34 to delimit the boundaries of the region containing significant linkage disequilibrium. Because of the nature of linkage disequilibrium between individual markers, not all markers within this region may be in significant LD.

### Analysis of Selection Line Allele Frequency Differences

The genotype results for each marker, for the two Parasite selection lines were tested for differences in allele frequency using the computer program Peddrift (Dodds and McEwan, 1997). This program estimates the likely distribution of allele frequencies between selection lines caused by random founder effects and genetic drift by simulation using the actual recorded pedigree structure. Significant divergence from the expected distribution is evidence of selection on a variant, near the genotyped marker, affecting the trait under selection: in this case fecal egg counts after a field challenge of gastrointestinal internal nematode parasites.

### Analysis of Marker Associations

Breeding values for the genotyped individuals were analyzed in the following manner. The EBVs for traits were adjusted for breed (even though EBVs had already been adjusted for this effect) with each marker allele fitted independently as a covariate (0=none, 1=one allele, 2=2 alleles) in a least squares model after the method of Fan et al. (2006). Fitting breed reduces the bias of admixture i.e. when the marker is associated with breed and true differences exist between breeds.

### Results

### Markers and Map Positions

The markers and their map positions are tabulated in Table 1. The BTA version 3.1 bovine genome assembly position given is 300 bp upstream of the actual dinucleotide repeat motif. The whole region is defined as including: the 300 bp upstream fragment, the dinucleotide repeat motif itself, and 300 bp downstream sequence. It was from this segment of DNA that the primers in Table 2 were designed. The markers are ordered in declining assembly order on bovine BTA11. The actual alleles observed and the length of their corresponding PCR products as measured by the ABI 3730 sequencer is tabulated in Table 3.

The IMF map positions are listed in centiMorgans (cM) defined from using a framework map starting from BMS1350 marker as 0 (not shown) and inserting the new BMS markers in their best location. Note some markers are unmapped and for some markers the order is not consistent with the bovine assembly order e.g. BMS1082722. The reasons for this are many. First the IMF resource can only reliably order markers greater than 5 cM apart. There also exists the possibility the bovine assembly is incorrect or that a genotyping error has occurred. Although in the latter case all apparent double recombinants have had their genotypes checked and where necessary eliminated. In other cases linkage mapping ordered markers that are not positioned in the current bovine genome assembly but can be ordered by linkage mapping.

The radiation hybrid map orders the markers in centiRays (cR) starting from zero, for BMS1082956. In theory this mapping technique should be more sensitive for ordering markers than linkage mapping in the IMF and appears to be able to discriminate and order markers where linkage mapping could not. However there were some apparent differences in order between the ovine and bovine genomes and it is not clear whether these are real or minor assembly and mapping discrepancies.

### Selection Lines Allele Frequency Differences

Table 4 lists the markers and the significance probability for an allele frequency difference between selection lines (resistant vs susceptible) after adjusting for founder effects and genetic drift. The assumption is that the allele frequencies have changed due to selection effects on a nearby locus in linkage disequilibrium with the measured marker. Four markers showed significant differences in allele frequency between selection lines in the Perendale flocks: BMS1784528, BMS1600436 BMS1081760 and BMS30480889. As shown later, all of these markers are located within the boundary defined by BMS1887400 to BMS1081770. In contrast no association was observed for any marker in the Romney selection lines.

WormFEC Sire Resource Association with Production and Host Resistance Traits

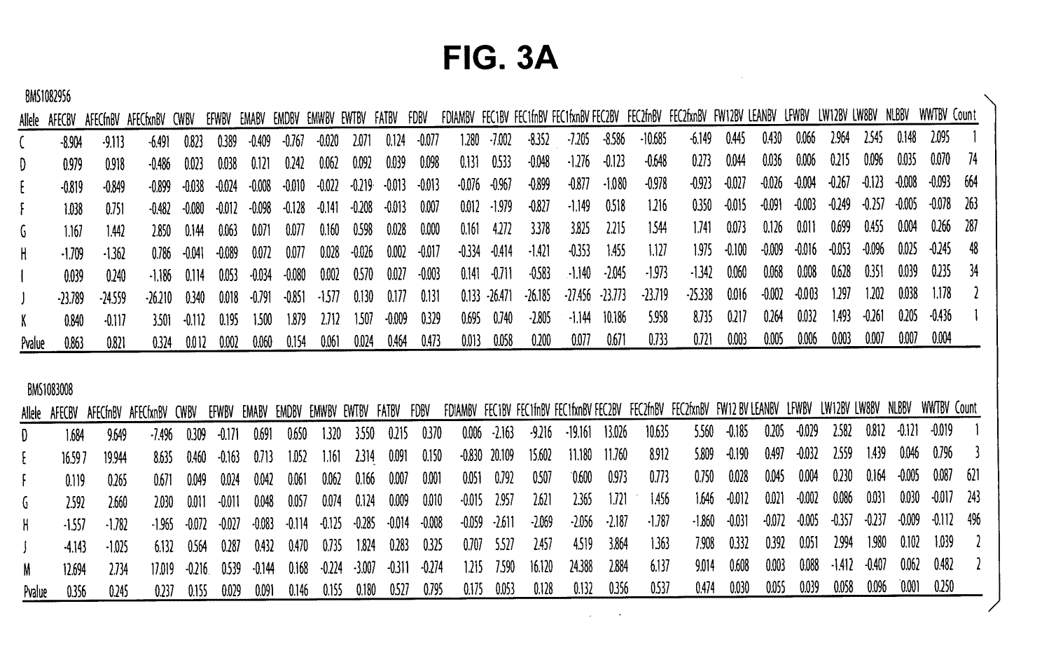

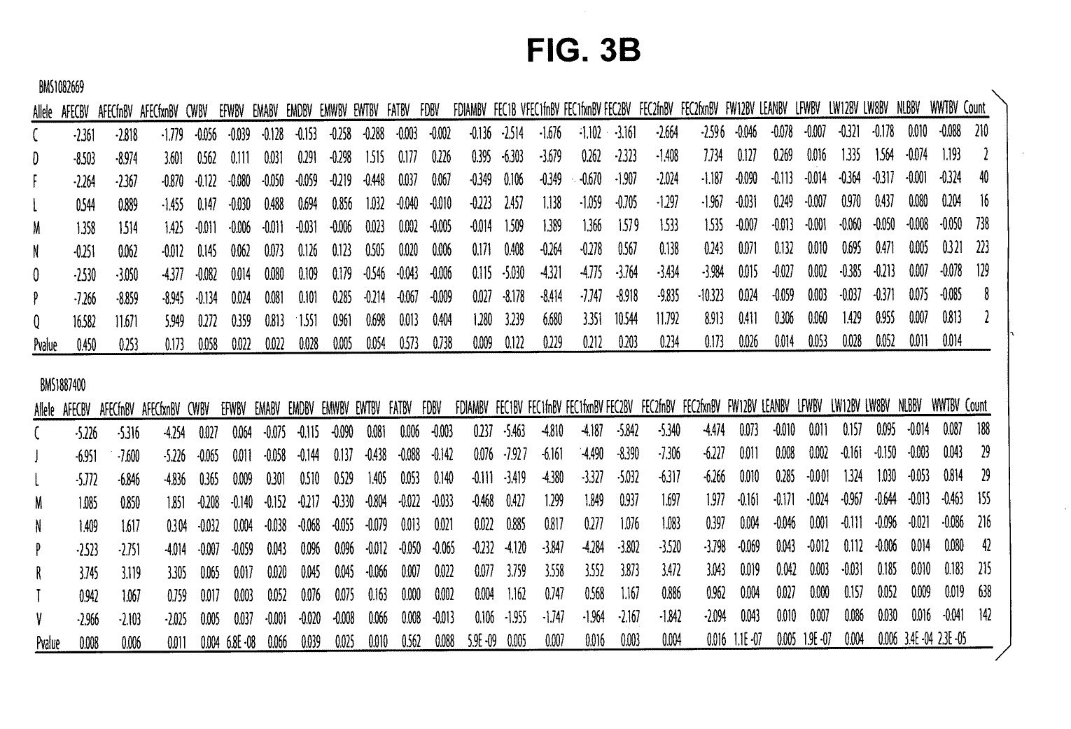

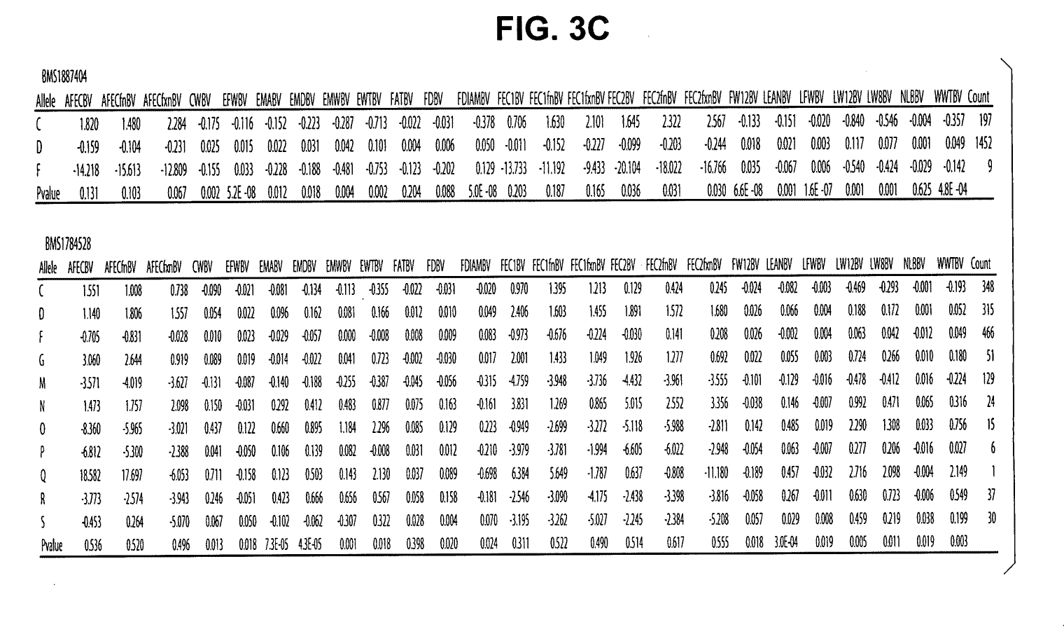

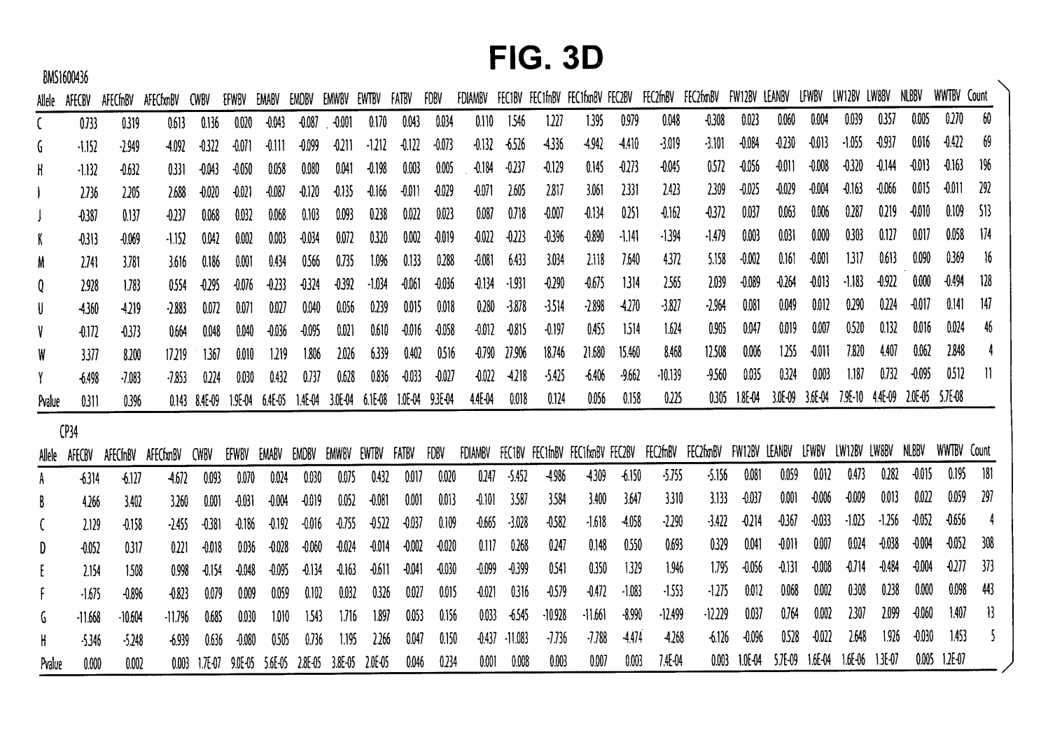

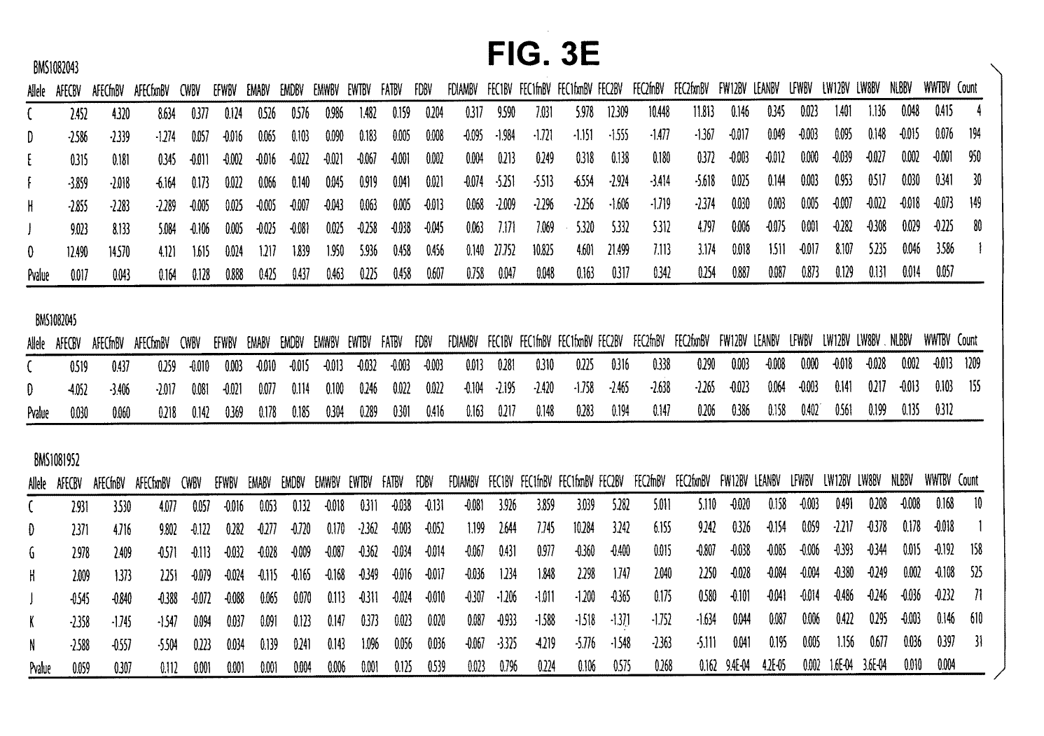

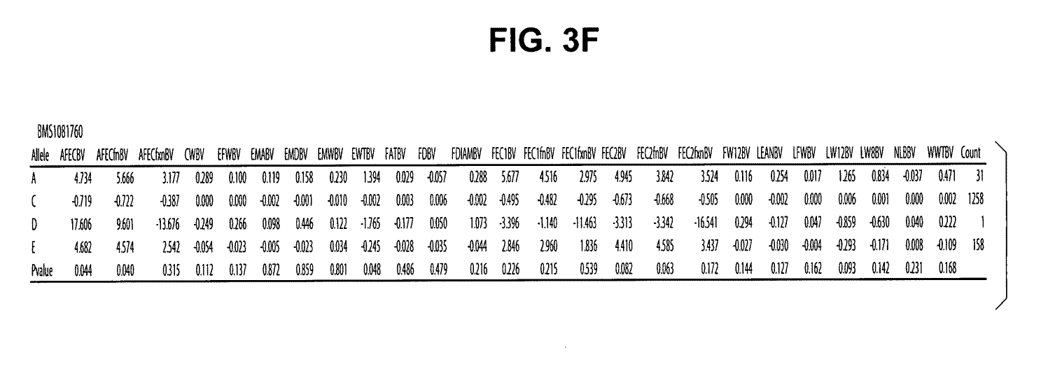

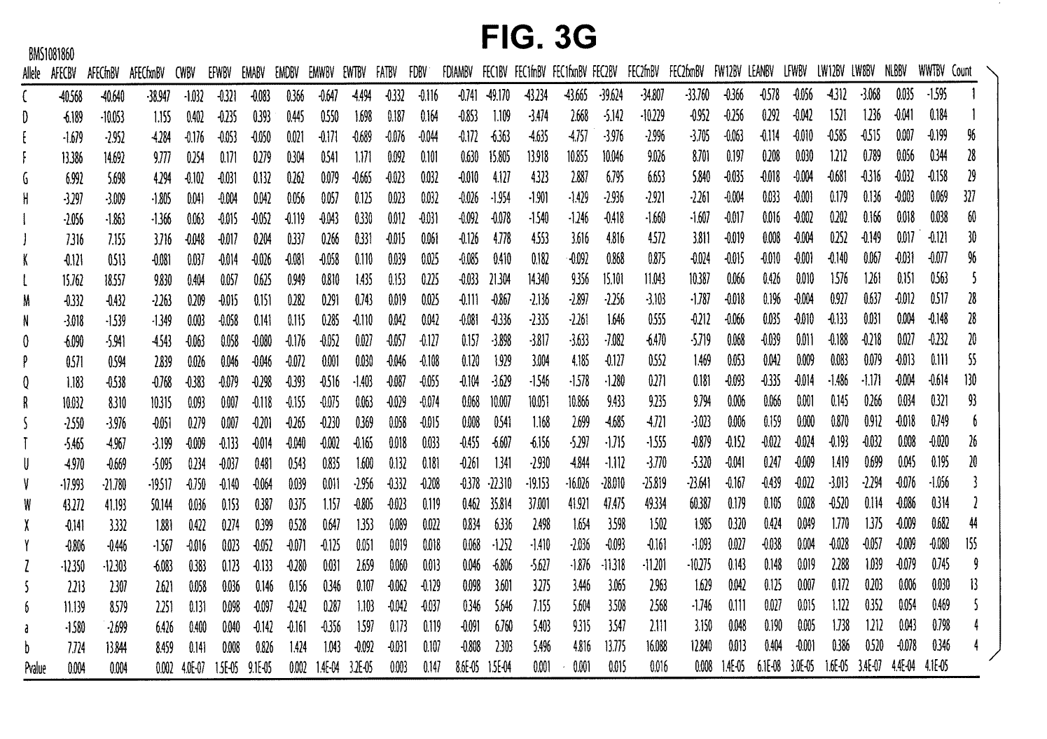

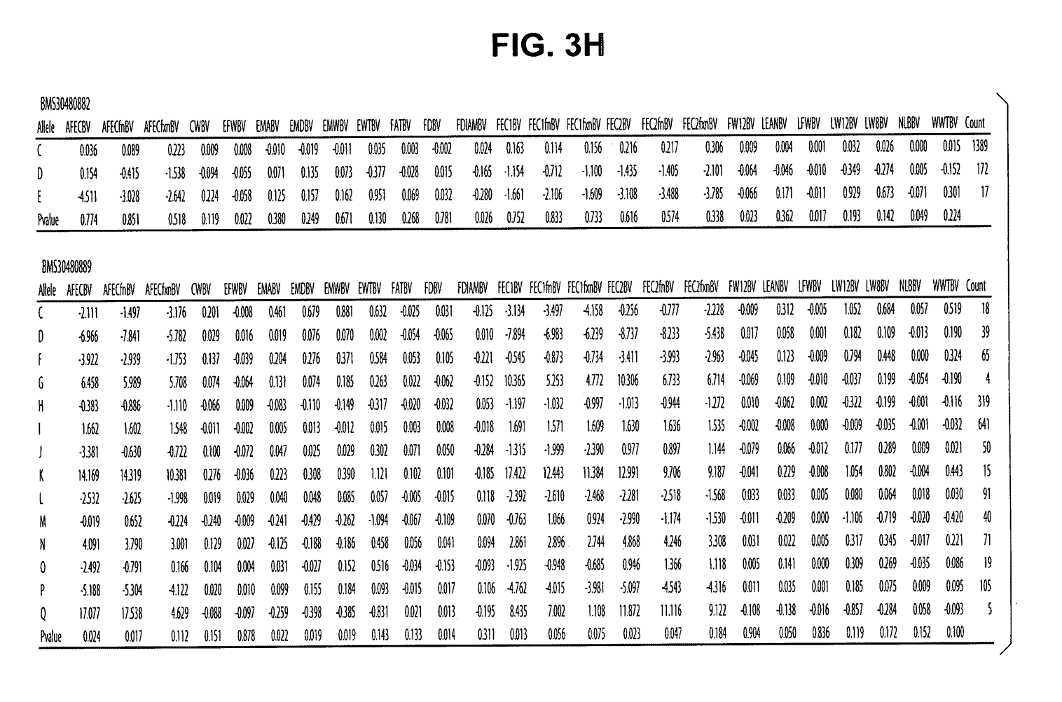

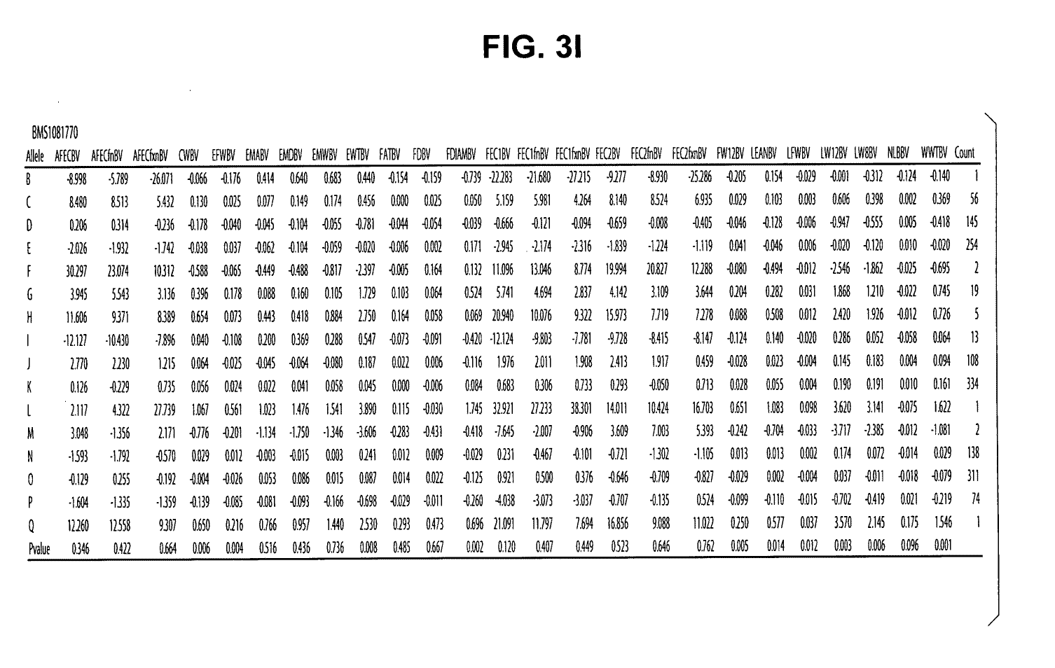

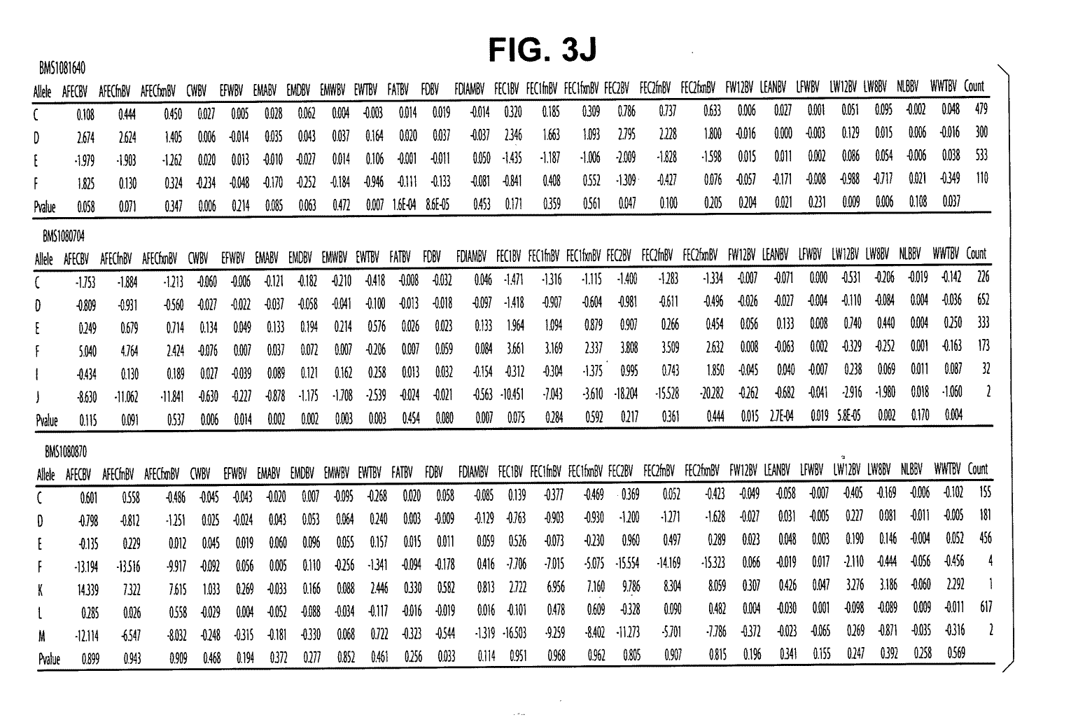

FIG. 3 lists for each marker tested for association in the WormFEC sire resource the various EBV allele estimates, their significance and the count of the alleles observed. All markers with the exception of BMS1080870 and BMS1082045 have allele significant associations (P<0.05) for more than one trait and within the boundary defined by BMS1887400 to BMS1081770 it is typically many traits. For example for CP34 it is 24 of the 25 traits listed.

Marker Linkage Disequilibrium with CP34

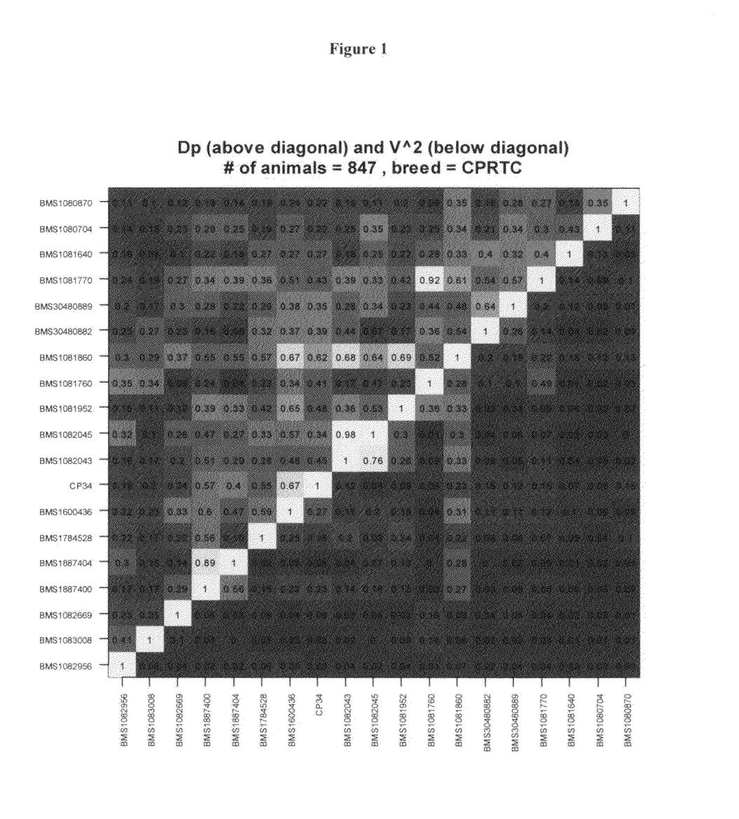

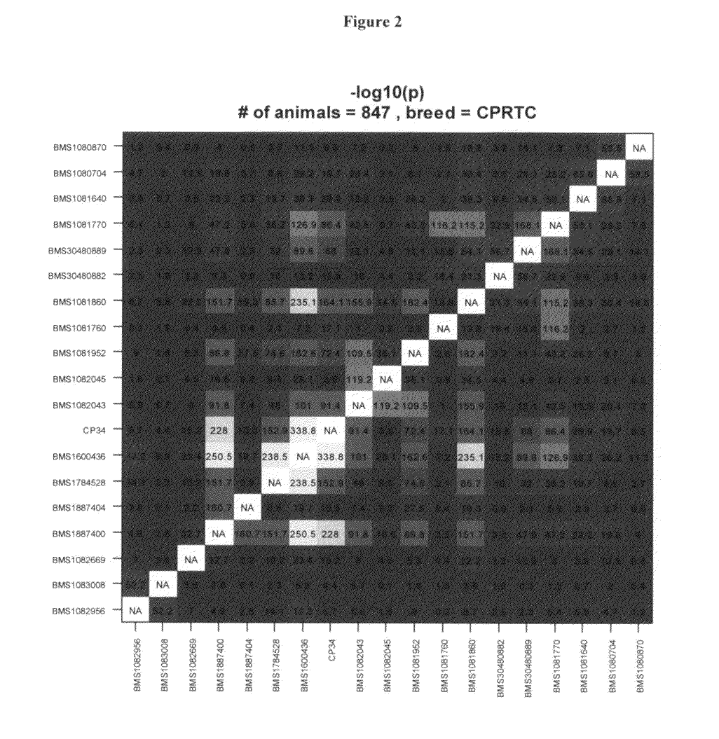

In the current context, markers with D′ values with CP34 greater than 0.3 or V² greater than 0.1 are considered to define the boundaries of useful LD, if they also have significant linkage disequilibrium with CP34. Based on the results presented in FIG. 1 and FIG. 2 this includes the region defined by BMS1887400 to BMS1081770. The linkage mapping distance between these 2 markers is 4.8 cM. Its estimated ovine genomic length, based on direct comparison with the bovine assembly, it is slightly greater than 1 million base pairs.

### Example of Predictive Ability of Markers in Industry Animals

The utility of the predictive ability is provided in the following example, but is not restricted solely to this approach. Selected CP34 trait estimated breeding value allele associations and their economic values (Young and Walker, 2007b) have been combined into an economic index in Table 5. The individual allele estimates are additive so an animal that has a genotype of AA will have, in the absence of other information, a predicted value of 66+66=132 cents versus an animal with a EE genotype of −81+−81=−162 cents. Used in this way individual animals can be ranked for breeding purposes. When estimated breeding values and their accuracies derived from trait measurements have been calculated, these marker based estimates can be blended to create an overall index using selection index theory and the relative accuracies of the two predictions. A further alternative is to fit the CP34 allele as either a fixed or random effect within the standard animal model BLUP evaluation.

Table 1 below, shows the map positions of the SSR markers identified.

TABLE 1

| | Bovine BTA11 | IMF position | RH position |
|---|---|---|---|
| Marker Name | position (Mbp) | Chr3 (cM) | Chr3 (cR) |
| BMS1084327 | 95234751 | 23.7 | unmapped |
| BMS1082942 | 93758528 | 25.1 | 25.2 |
| BMS1082956 | 93726770 | 25.1 | 0 |
| BMS1082961 | 93712544 | 25.1 | 0 |
| BMS1083945 | 93639984 | 25.1 | 25.2 |
| BMS1083008 | 93473429 | 25.1 | 0 |
| BMS1082252 | 89948180 | unmapped | 37.9 |
| BMS1082669 | 88338219 | 30.4 | 65.5 |
| BMS1082702 | 88245390 | unmapped | unmapped |
| BMS1082722 | 88195495 | 29.4 | 65.5 |
| BMS1082831 | 87126578 | 32.3 | unmapped |
| BMS1887400 | 86996579 | 33 | unmapped |
| BMS1887404 | 86985063 | 33 | unmapped |
| BMS1784528 | 86739524 | 33 | unmapped |
| BMS1600436 | 86671265 | 33.9 | 106.6 |
| CP34 | 86655663 | 33.9 | 106.6 |
| BMS1082043 | 86431911 | 35.2 | 98.5 |
| BMS1082045 | 86438829 | 35.8 | 128.5 |
| BMS1081952 | 86251004 | 35.8 | 101.2 |
| BMS1081760 | 86017933 | 35.8 | unmapped |
| BMS1081860 | ChrUn.003.1357 | 35.8 | 128.5 |
| BMS30480882 | 85850308 | 37.7 | unmapped |
| BMS30480889 | 85863541 | 37.7 | unmapped |
| BMS1081770 | 85981658 | 37.8 | unmapped |
| BMS1081774 | 85964979 | 37.8 | unmapped |
| RSAD2_1 | 85650772 | 38.9 | 135.7 |
| BMS1081640 | ~85442209 | 41.1 | 89.9 |
| BMS1080704 | 84881378 | 43.2 | unmapped |
| BMS1080870 | 71477314 | 43.6 | unmapped |

* map positions refer to the relative start positions of the SSR

Table 2, below shows the primer sequences used to amplify the SSR markers identified.

TABLE 2

| | | | SEQ | (SSR |
|---|---|---|---|---|
| Marker name | Primer Sequences | | ID NO: | Motif)n |
| BMS1084327 | Fwd | TGTAAAACGACGGCCAGTTTCCTTCCCCAGACAGTCAC | 1 | AC |
| | Rev | GTTTCTTTGTGTATTTGGGAGGGGTGT | 2 | |
| BMS1082942 | Fwd | TGTAAAACGACGGCCAGTCATGTGTGTCAACATCAATCCA | 3 | AC |
| | Rev | GTTTCTTTCCATCCAGACAATACAGCAA | 4 | |
| BMS1082956 | Fwd | TGTAAAACGACGGCCAGTACTGGTCAAGCAGACCATCT | 5 | AC |
| | Rev | GTTTCTTCCCATGTTCAGGCGTTATCT | 6 | |
| BMS1082961 | Fwd | TGTAAAACGACGGCCAGTGGCAGGTGAAAATACTTGCTG | 7 | AC |
| | Rev | GTTTCTTTGATGAGGCAGCTCATTGAC | 8 | |
| BMS1083945 | Fwd | TGTAAAACGACGGCCAGTCAAGATGAATGATCCCATGC | 9 | AG |
| | Rev | GTTTCTTTCAGCCCAGGAGTTAAACATT | 10 | |
| BMS1083008 | Fwd | TGTAAAACGACGGCCAGTGTCCTCTCAGATGGCAGAGC | 11 | AC |
| | Rev | GTTTCTTTGGAGACATTAGTGTGTGCTCAT | 12 | |
| BMS1082252 | Fwd | TGTAAAACGACGGCCAGTATGGTCACCACTGCACTGAC | 13 | AC |
| | Rev | GTTTCTTAAGGCAGGCAAGTATTTGGA | 14 | |
| BMS1082669 | Fwd | TGTAAAACGACGGCCAGTGGGGAGTATGCAATTCAGGA | 15 | AC |
| | Rev | GTTTCTTTACAGGCCAAAGGGAATTTG | 16 | |
| BMS1082702 | Fwd | TGTAAAACGACGGCCAGTGCGTGTGGATAGCGTGAGTA | 17 | AC |
| | Rev | GTTTCTTTTGAGACCCCAGTCCAGAAG | 18 | |
| BMS1082722 | Fwd | TGTAAAACGACGGCCAGTGGATATCAGGGAGTGGGATG | 19 | AC |
| | Rev | GTTTCTTTCCCCTGATGTTAGCAGCTT | 20 | |
| BMS1082831 | Fwd | TGTAAAACGACGGCCAGTCTGCTCCATATCACGACAGC | 21 | AC |
| | Rev | GTTTCTTTGGTCTTGGTGGTCTGTTTG | 22 | |
| BMS1887400 | Fwd | TGTAAAACGACGGCCAGTGAAAGGTGGTGGTCTCCTTG | 23 | AC |
| | Rev | GTTTCTTTGAGAGAAGACCTGGGGAGA | 24 | |
| BMS1887404 | Fwd | TGTAAAACGACGGCCAGTGGGTCGTAGAGAGTTGAACACA | 25 | AC |
| | Rev | GTTTCTTGCTGTCTCTTTCACTCCAAAATC | 26 | |
| BMS1784528 | Fwd | TGTAAAACGACGGCCAGTCTCTGAGCCATATGGGAAGC | 27 | AT |
| | Rev | GTTTCTTTTCCACAGTGTTTCAGATGTATAGC | 28 | |
| BMS1600436 | Fwd | TGTAAAACGACGGCCAGTTCAGGAAGTGGTAGGCAGAGA | 29 | AC |
| | Rev | GTTTCTTTACCACTGAGCCACCAGAGA | 30 | |
| CP34 | Fwd | TGTAAAACGACGGCCAGTGCTGAACAATGTGATATGTTCAGG | 31 | AC |
| | Rev | GTTTCTTGGGACAATACTGTCTTAGATGCTGC | 32 | |
| BMS1082043 | Fwd | TGTAAAACGACGGCCAGTGGGAAACCCACACAACAGAG | 33 | AC |
| | Rev | GTTTCTTGGAGAATGGCATGGACAGAG | 34 | |
| BMS1082045 | Fwd | TGTAAAACGACGGCCAGTTTCTTCAGCACTCAGCCTTCT | 35 | AC |
| | Rev | GTTTCTTTCACTGCTGGATATGGTGGA | 36 | |
| BMS1081952 | Fwd | TGTAAAACGACGGCCAGTTTGCAAGGTTAGACTTTGGTGA | 37 | AC |
| | Rev | GTTTCTTTGTTCCCAGACCAGTATTTCAG | 38 | |
| BMS1081760 | Fwd | TGTAAAACGACGGCCAGTCTCAAAACGACAAAGCCACA | 39 | AT |
| | Rev | GTTTCTTAGGACCGGCTGTATAGCACA | 40 | |
| BMS1081860 | Fwd | TGTAAAACGACGGCCAGTGCAGGCTGGTTCTTTACCAC | 41 | AC |
| | Rev | GTTTCTTTTGTGGTAGGTTTCACCAAGG | 42 | |
| BMS30480882 | Fwd | TGTAAAACGACGGCCAGTGCAAATGGCCAAATGTCATC | 43 | AT |
| | Rev | GTTTCTTCATGCACCCCAATGTTCATA | 44 | |
| BMS30480889 | Fwd | TGTAAAACGACGGCCAGTTCTTGATCACTGAGCCACCA | 45 | AC |
| | Rev | GTTTCTTTCAGCAAAGAGGCTGGTACA | 46 | |
| BMS1081770 | Fwd | TGTAAAACGACGGCCAGTAAAGCGTTGCTATCTGTCACAA | 47 | AC |
| | Rev | GTTTCTTGCTGTCCTGAGCACATAGGG | 48 | |
| BMS1081774 | Fwd | TGTAAAACGACGGCCAGTTGGAATCCCTTGGACAGAAC | 49 | AC |
| | Rev | GTTTCTTCCCTGACTCCTAATGCCATC | 50 | |
| RSAD2_1 | Fwd | TGTAAAACGACGGCCAGTTAGCAAACATGTGGGTGGTC | 51 | AC |
| | Rev | GTTTCTTTTTGCAGAGCCGTATTTGTG | 52 | |
| BMS1081640 | Fwd | TGTAAAACGACGGCCAGTTTTTAGGTGTACAGCAGAGTGATG | 53 | AT |
| | Rev | GTTTCTTGGAGGCTTGGTGTGCTACAG | 54 | |
| BMS1080704 | Fwd | TGTAAAACGACGGCCAGTACTCACCCTGAGTGCTCCAC | 55 | AC |
| | Rev | GTTTCTTCTCCGGGGTTTCTCTTCTCT | 56 | |
| BMS1080870 | Fwd | TGTAAAACGACGGCCAGTAATGGGGCAGCAAAGAGTT | 57 | AC |
| | Rev | GTTTCTTCTCCGGGGTTTCTCTTCTCT | 58 | |

*M13 sequence indicated in italic font (TGTAAAACGACGGCCAGT-SEQ ID NO:59)
Pigtail sequence indicated in italic font (GTTTCTT-SEQ ID NO:60)
Sequence not in italics, represents sequence of the ovine genome flanking the SSR marker and therefore is specific for the particular SSR locus

Table 3, below shows a summary of the allele information for the SSR markers identified.

TABLE 3

| | | | | | Number |
|---|---|---|---|---|---|
| | No. of | Bin | Fragment sizes | Bin length | alleles |
| Marker name | alleles | name | (bp) | (bp) | CPRTC |
| BMS1084327 | 2 | C | 177.5 +/− 0.7 | | |
| | | D | 179.5 +/− 0.7 | 2 | |
| BMS1082942 | 6 | C | 183.9 +/− 0.7 | | |
| | | D | 186.1 +/− 0.7 | 2.2 | |
| | | F | 190.1 +/− 0.7 | | |
| | | G | 192.2 +/− 0.7 | 2.1 | |
| | | H | 194.3 +/− 0.7 | 2.1 | |
| | | I | 196.4 +/− 0.7 | 2.1 | |
| BMS1082956 | 9 | C | 175.4 +/− 0.7 | | 1 |
| | | D | 177.6 +/− 0.7 | 2.2 | 74 |
| | | E | 179.3 +/− 0.7 | 1.7 | 664 |
| | | F | 181.3 +/− 0.7 | 2 | 263 |
| | | G | 183.2 +/− 0.7 | 1.9 | 287 |
| | | H | 185.0 +/− 0.7 | 1.8 | 48 |
| | | I | 186.9 +/− 0.7 | 1.9 | 34 |
| | | J | 188.7 +/− 0.7 | 1.8 | 2 |
| | | K | 190.5 +/− 0.7 | 1.8 | 1 |
| BMS1082961 | 2 | C | 176.1 +/− 0.7 | | |
| | | D | 178.0 +/− 0.7 | 1.9 | |
| BMS1083945 | 3 | C | 154.6 +/− 0.7 | | |
| | | G | 162.6 +/− 0.7 | | |
| | | H | 164.5 +/− 0.7 | 1.9 | |
| BMS1083008 | 10 | C | 171.8 +/− 0.45 | | |
| | | D | 173.9 +/− 0.45 | 2.1 | 1 |
| | | E | 175.7 +/− 0.45 | 1.8 | 3 |
| | | F | 187.4 +/− 0.45 | | 621 |
| | | G | 188.7 +/− 0.45 | 1.3 | 243 |
| | | H | 190.4 +/− 0.45 | 1.7 | 496 |
| | | J | 193.2 +/− 0.45 | | 2 |
| | | K | 195.0 +/− 0.45 | 1.8 | |
| | | L | 196.9 +/− 0.45 | 1.9 | 2 |
| | | M | 204.4 +/− 0.45 | | |
| BMS1082252 | 3 | C | 161.6 +/− 0.7 | | |
| | | D | 163.5 +/− 0.7 | 1.9 | |
| | | E | 165.4 +/− 0.7 | 1.9 | |
| BMS1082669 | 10 | C | 183.7 +/− 0.7 | | 210 |
| | | D | 185.6 +/− 0.7 | 1.9 | 2 |
| | | F | 189.6 +/− 0.7 | | 40 |
| | | J | 197.1 +/− 0.7 | | |
| | | L | 200.9 +/− 0.7 | | 16 |
| | | M | 202.7 +/− 0.7 | 1.8 | 738 |
| | | N | 204.7 +/− 0.7 | 2 | 223 |
| | | O | 206.5 +/− 0.7 | 1.8 | 129 |
| | | P | 208.4 +/− 0.7 | 1.9 | 8 |
| | | Q | 210.3 +/− 0.7 | 1.9 | 2 |
| BMS1082702 | 9 | C | 199.5 +/− 0.45 | | |
| | | G | 203.7 +/− 0.45 | | |
| | | I | 205.6 +/− 0.45 | | |
| | | K | 207.7 +/− 0.45 | | |
| | | L | 213.8 +/− 0.45 | | |
| | | N | 216.1 +/− 0.41 | | |
| | | O | 217.0 +/− 0.41 | 0.9 | |
| | | Q | 219.1 +/− 0.45 | | |
| | | S | 221.1 +/− 0.45 | | |
| BMS1082722 | 7 | C | 172.6 +/− 0.7 | | |
| | | E | 176.6 +/− 0.7 | | |
| | | G | 181.0 +/− 0.7 | | |
| | | H | 182.9 +/− 0.7 | 1.9 | |
| | | I | 185.1 +/− 0.7 | 2.2 | |
| | | L | 191.3 +/− 0.7 | | |
| | | P | 199.7 +/− 0.7 | | |
| BMS1082831 | 10 | C | 172.9 +/− 0.7 | | |
| | | D | 174.8 +/− 0.7 | 1.9 | |
| | | E | 188.1 +/− 0.7 | | |
| | | F | 190.0 +/− 0.7 | 1.9 | |
| | | G | 192.0 +/− 0.7 | 2 | |
| | | H | 193.9 +/− 0.7 | 1.9 | |
| | | M | 203.4 +/− 0.7 | | |
| | | N | 205.2 +/− 0.7 | 1.8 | |
| | | Q | 211.0 +/− 0.7 | | |
| | | R | 212.7 +/− 0.7 | 1.7 | |
| BMS1887400 | 10 | C | 196.1 +/− 0.45 | | 188 |
| | | J | 209.4 +/− 0.45 | | 29 |
| | | L | 211.2 +/− 0.43 | | 29 |
| | | M | 212.1 +/− 0.42 | 0.9 | 155 |
| | | N | 213.1 +/− 0.42 | 1 | 216 |
| | | P | 215.0 +/− 0.45 | | 42 |
| | | R | 216.9 +/− 0.45 | | 215 |
| | | T | 218.8 +/− 0.45 | | 638 |
| | | V | 220.7 +/− 0.45 | | 142 |
| | | X | 222.6 +/− 0.45 | | |
| BMS1887404 | 5 | C | 217.2 +/− 0.7 | | 197 |
| | | D | 218.9 +/− 0.7 | 1.7 | 1452 |
| | | E | 221.1 +/− 0.7 | 2.2 | |
| | | F | 222.8 +/− 0.7 | 1.7 | 9 |
| | | H | 226.6 +/− 0.7 | | |
| BMS1784528 | 12 | C | 161.3 +/− 0.45 | | 348 |
| | | D | 163.3 +/− 0.45 | 2 | 315 |
| | | F | 166.0 +/− 1.46 | | 466 |
| | | G | 168.7 +/− 0.45 | 2.7 | 51 |
| | | L | 178.7 +/− 0.45 | | |
| | | M | 180.7 +/− 0.45 | 2 | 129 |
| | | N | 182.6 +/− 0.45 | 1.9 | 24 |
| | | O | 184.7 +/− 0.45 | 2.1 | 15 |
| | | P | 186.6 +/− 0.45 | 1.9 | 6 |
| | | Q | 188.7 +/− 0.45 | 2.1 | 1 |
| | | R | 190.3 +/− 0.45 | 1.6 | 37 |
| | | S | 192.2 +/− 0.45 | 1.9 | 30 |
| BMS1600436 | 15 | C | 178.6 +/− 0.7 | | 60 |
| | | G | 187.2 +/− 0.7 | | 69 |
| | | H | 189.2 +/− 0.7 | 2 | 196 |
| | | I | 191.3 +/− 0.7 | 2.1 | 292 |
| | | J | 193.3 +/− 0.7 | 2 | 513 |
| | | K | 195.5 +/− 0.7 | 2.2 | 174 |
| | | M | 199.5 +/− 0.7 | | 16 |
| | | P | 205.4 +/− 0.7 | | |
| | | Q | 207.5 +/− 0.7 | 2.1 | 128 |
| | | T | 213.2 +/− 0.7 | | |
| | | U | 215.3 +/− 0.7 | 2.1 | 147 |
| | | V | 217.3 +/− 0.7 | 2 | 46 |
| | | W | 219.8 +/− 0.7 | 2.5 | 4 |
| | | X | 221.6 +/− 0.7 | 1.8 | |
| | | Y | 223.4 +/− 0.7 | 1.8 | 11 |
| CP34 | 8 | F | 134.3 +/− 0.7 | | 443 |
| | | E | 136.3 +/− 0.7 | 2 | 373 |
| | | D | 138.3 +/− 0.7 | 2 | 308 |
| | | C | 140.4 +/− 0.7 | 2.1 | 4 |
| | | B | 142.5 +/− 0.7 | 2.1 | 297 |
| | | A | 144.6 +/− 0.7 | 2.1 | 181 |
| | | G | 146.7 +/− 0.7 | 2.1 | 13 |
| | | H | 148.8 +/− 0.7 | 2.1 | 5 |
| BMS1082043 | 9 | C | 148.0 +/− 0.7 | | 4 |
| | | D | 150.3 +/− 0.7 | 2.3 | 194 |
| | | E | 152.5 +/− 0.7 | 2.2 | 950 |
| | | F | 154.6 +/− 0.7 | 2.1 | 30 |
| | | H | 158.9 +/− 0.7 | | 149 |
| | | J | 163.2 +/− 0.7 | | 80 |
| | | K | 165.2 +/− 0.7 | 2 | |
| | | M | 169.2 +/− 0.7 | | |
| | | O | 173.9 +/− 0.7 | | 1 |
| BMS1082045 | 2 | C | 160.1 +/− 0.7 | | 1209 |
| | | D | 162.0 +/− 0.7 | 1.9 | 155 |
| BMS1081952 | 7 | C | 181.7 +/− 0.7 | | 10 |
| | | D | 183.8 +/− 0.7 | 2.1 | 1 |
| | | G | 189.9 +/− 0.7 | | 158 |
| | | H | 192.0 +/− 0.7 | 21 | 525 |
| | | J | 196.1 +/− 0.7 | | 71 |
| | | K | 198.2 +/− 0.7 | 2.1 | 610 |
| | | N | 204.3 +/− 0.7 | | 31 |
| BMS1081760 | 5 | A | 170.5 +/− 0.7 | | 31 |
| | | B | 172.5 +/− 0.7 | 2 | |
| | | C | 174.5 +/− 0.7 | 2 | 1258 |
| | | D | 176.5 +/− 0.7 | 2 | 1 |
| | | E | 178.5 +/− 0.7 | 2 | 158 |
| BMS1081860 | 29 | C | 204.5 +/− 0.7 | | 1 |
| | | D | 206.5 +/− 0.7 | 2 | 1 |
| | | E | 208.3 +/− 0.7 | 1.8 | 96 |
| | | F | 210.3 +/− 0.7 | 2 | 28 |
| | | G | 212.2 +/− 0.7 | 1.9 | 29 |
| | | H | 214.0 +/− 0.7 | 1.8 | 327 |
| | | I | 215.8 +/− 0.7 | 1.8 | 60 |
| | | J | 217.7 +/− 0.7 | 1.9 | 30 |
| | | K | 219.5 +/− 0.7 | 1.8 | 96 |
| | | L | 221.5 +/− 0.7 | 2 | 5 |
| | | M | 223.4 +/− 0.7 | 1.9 | 28 |
| | | N | 225.2 +/− 0.7 | 1.8 | 28 |
| | | O | 227.1 +/− 0.7 | 1.9 | 20 |
| | | P | 229.0 +/− 0.7 | 1.9 | 55 |
| | | Q | 230.9 +/− 0.7 | 1.9 | 130 |
| | | R | 232.7 +/− 0.7 | 1.8 | 93 |
| | | S | 234.6 +/− 0.7 | 1.9 | 6 |
| | | T | 236.5 +/− 0.7 | 1.9 | 26 |
| | | U | 238.4 +/− 0.7 | 1.9 | 20 |
| | | V | 240.4 +/− 0.7 | 2 | 3 |
| | | W | 242.4 +/− 0.7 | 2 | 2 |
| | | X | 244.2 +/− 0.7 | 1.8 | 44 |
| | | Y | 246.2 +/− 0.7 | 2 | 155 |
| | | Z | 248.0 +/− 0.7 | 1.8 | 9 |
| | | 5 | 259.4 +/− 0.7 | | 13 |
| | | 6 | 261.4 +/− 0.7 | 2 | 5 |
| | | 7 | 263.3 +/− 0.7 | 1.9 | |
| | | a | 269.0 +/− 0.7 | | 4 |
| | | b | 271.0 +/− 0.7 | 2 | 4 |
| BMS30480882 | 3 | C | 198.4 +/− 0.7 | | 1389 |
| | | D | 200.3 +/− 0.7 | 1.9 | 172 |
| | | E | 202.3 +/− 0.7 | 2 | 17 |
| BMS30480889 | 16 | C | 188.8 +/− 0.7 | | 18 |
| | | D | 190.7 +/− 0.7 | 1.9 | 39 |
| | | E | 192.7 +/− 0.7 | 2 | |
| | | F | 194.6 +/− 0.7 | 1.9 | 65 |
| | | G | 196.7 +/− 0.7 | 2.1 | 4 |
| | | H | 198.6 +/− 0.7 | 1.9 | 319 |
| | | I | 200.5 +/− 0.7 | 1.9 | 641 |
| | | J | 202.3 +/− 0.7 | 1.8 | 50 |
| | | K | 204.2 +/− 0.7 | 1.9 | 15 |
| | | L | 206.1 +/− 0.7 | 1.9 | 91 |
| | | M | 208.3 +/− 0.7 | 2.2 | 40 |
| | | N | 210.4 +/− 0.7 | 2.1 | 71 |
| | | O | 212.4 +/− 0.7 | 2 | 19 |
| | | P | 214.5 +/− 0.7 | 2.1 | 105 |
| | | Q | 216.5 +/− 0.7 | 2 | 5 |
| | | R | 224.1 +/− 0.7 | | |
| BMS1081770 | 16 | B | 213.8 +/− 0.45 | | 1 |
| | | C | 215.6 +/− 0.45 | 1.8 | 56 |
| | | D | 217.5 +/− 0.45 | 1.9 | 145 |
| | | E | 219.5 +/− 0.45 | 2 | 254 |
| | | F | 221.4 +/− 0.45 | 1.9 | 2 |
| | | G | 223.2 +/− 0.45 | 1.8 | 19 |
| | | H | 225.2 +/− 0.45 | 2 | 5 |
| | | I | 227.0 +/− 0.45 | 1.8 | 13 |
| | | J | 228.8 +/− 0.45 | 1.8 | 108 |
| | | K | 230.3 +/− 0.45 | 1.5 | 334 |
| | | L | 232.1 +/− 0.45 | 1.8 | 1 |
| | | M | 234.5 +/− 0.45 | 2.4 | 2 |
| | | N | 236.4 +/− 0.45 | 1.9 | 138 |
| | | O | 238.3 +/− 0.45 | 1.9 | 311 |
| | | P | 240.2 +/− 0.45 | 1.9 | 74 |
| | | Q | 242.2 +/− 0.45 | 2 | 1 |
| BMS1081774 | 6 | C | 184.4 +/− 0.7 | | |
| | | D | 186.6 +/− 0.7 | 2.2 | |
| | | E | 188.6 +/− 0.7 | 2 | |
| | | F | 190.8 +/− 0.7 | 2.2 | |
| | | H | 194.8 +/− 0.7 | | |
| | | I | 196.9 +/− 0.7 | 2.1 | |
| RSAD2_1 | 2 | C | 189.7 +/− 0.7 | | |
| | | D | 191.7 +/− 0.7 | 2 | |
| BMS1081640 | 4 | C | 144.8 +/− 0.7 | | 479 |
| | | D | 147.1 +/− 0.7 | 2.3 | 300 |
| | | E | 149.3 +/− 0.7 | 2.2 | 533 |
| | | F | 151.5 +/− 0.7 | 2.2 | 110 |
| BMS1080704 | 8 | C | 182.1 +/− 0.7 | | 226 |
| | | D | 184.2 +/− 0.7 | 2.1 | 652 |
| | | E | 186.3 +/− 0.7 | 2.1 | 333 |
| | | F | 188.4 +/− 0.7 | 2.1 | 173 |
| | | G | 190.5 +/− 0.7 | 2.1 | |
| | | H | 192.6 +/− 0.7 | 2.1 | |
| | | I | 194.6 +/− 0.7 | 2 | 32 |
| | | J | 196.7 +/− 0.7 | 2.1 | 2 |
| BMS1080870 | 7 | C | 156.0 +/− 0.7 | | 155 |
| | | D | 158.0 +/− 0.7 | 2 | 181 |
| | | E | 160.1 +/− 0.7 | 2.1 | 456 |
| | | F | 161.9 +/− 0.7 | 1.8 | 4 |
| | | K | 171.6 +/− 0.7 | | 1 |
| | | L | 173.5 +/− 0.7 | 1.9 | 617 |
| | | M | 175.6 +/− 0.7 | 2.1 | 2 |

Table 4, below shows Peddrift results by selection line expressed as −log 10 (significance probability).

TABLE 4

| Marker Name | RSL | PSL |
|---|---|---|
| BMS1084327 | | |
| BMS1082942 | | |
| BMS1082956 | 0.208 | 0.237 |
| BMS1082961 | | |
| BMS1083945 | | |
| BMS1083008 | 0.456 | 0.325 |
| BMS1082252 | | |
| BMS1082669 | 0.432 | 0.263 |
| BMS1082702 | | |
| BMS1082722 | | |
| BMS1082831 | | |
| BMS1887400 | 0.340 | 0.753 |
| BMS1887404 | mono | 0.785 |
| BMS1784528 | 0.228 | 1.3224 |
| BMS1600436 | 0.412 | 3.1549 |
| CP34 | 0.842 | 0.799 |
| BMS1082043 | 0.403 | 0.068 |
| BMS1082045 | 0.080 | 0.112 |
| BMS1081952 | 0.394 | 0.334 |
| BMS1081760 | 0.347 | 1.3401 |
| BMS1081860 | 0.236 | 0.412 |
| BMS30480882 | 0.507 | 0.223 |
| BMS30480889 | 0.011 | 1.367 |
| BMS1081770 | 0.010 | 1.1238 |
| BMS1081774 | | |
| RSAD2_1 | | |
| BMS1081640 | 0.215 | 0.636 |
| BMS1080704 | 0.577 | 0.652 |
| BMS1080870 | 0.282 | 1.161 |

* −log10(p) values
RSL: Romney Selection Line
PSL: Perendale Selection Line

Allele effects by marker and significance are shown in FIG. 3.

Table 5 below shows allele estimates for CP34 for the BV traits analyzed for the Romney, Coopworth, Perendale, Texel and Composite analysis in their standard SIL trait units coupled with combined standard SIL economic estimates in cents for: growth adjusted for meat value (Gm), Meat value adjusted for growth (Mg), wool, Number of lambs born/ewe wintered (NLB), and combined host resistance (FEC) plus their additive overall index sum. Significance values for each trait are listed at the bottom (* P<0.05, ** P<0.01, *** P<0.001)

TABLE 5

| allele | no. | WWTBV | EWTBV | CWBV | LFWBV | FW12BV | EFWBV | LEANBV | FATBV |
|---|---|---|---|---|---|---|---|---|---|
| A | 180 | 0.20 | 0.43 | 0.09 | 0.01 | 0.08 | 0.07 | 0.06 | 0.02 |
| B | 297 | 0.06 | −0.08 | 0.00 | −0.01 | −0.04 | −0.03 | 0.00 | 0.00 |
| C | 4 | −0.66 | −0.52 | −0.38 | −0.03 | −0.21 | −0.19 | −0.37 | −0.04 |
| D | 307 | −0.05 | −0.01 | −0.02 | 0.01 | 0.04 | 0.04 | −0.01 | 0.00 |
| E | 373 | −0.28 | −0.61 | −0.15 | −0.01 | −0.06 | −0.05 | −0.13 | −0.04 |
| F | 443 | 0.10 | 0.33 | 0.08 | 0.00 | 0.01 | 0.01 | 0.07 | 0.03 |
| G | 13 | 1.41 | 1.90 | 0.69 | 0.00 | 0.04 | 0.03 | 0.76 | 0.05 |
| H | 5 | 1.45 | 2.27 | 0.64 | −0.02 | −0.10 | −0.08 | 0.53 | 0.05 |
| sign | | *** | *** | *** | ** | *** | *** | *** | * |

| allele | NLBBV | FEC1BV | FEC2BV | AFECBV | Gm | Mg | wool | NLB | Fec | index |
|---|---|---|---|---|---|---|---|---|---|---|
| A | −0.01 | −5.45 | −6.15 | −6.32 | 5 | 14 | 34 | −36 | 49 | 66 |
| B | 0.02 | 3.58 | 3.64 | 4.26 | 13 | 0 | −16 | 54 | −32 | 19 |
| C | −0.05 | −3.03 | −4.06 | 2.13 | −92 | −101 | −91 | −126 | 15 | −394 |
| D | 0.00 | 0.26 | 0.55 | −0.06 | −7 | −3 | 18 | −11 | −2 | −5 |
| E | 0.00 | −0.40 | 1.33 | 2.15 | −10 | −31 | −24 | −9 | −8 | −81 |
| F | 0.00 | 0.31 | −1.09 | −1.68 | −1 | 15 | 5 | 0 | 6 | 25 |
| G | −0.06 | −6.55 | −8.99 | −11.67 | 123 | 214 | 14 | −145 | 74 | 279 |
| H | −0.03 | −11.09 | −4.48 | −5.35 | 94 | 146 | −43 | −72 | 59 | 184 |
| sign | ** | ** | ** | *** | | | | | | |

The markers and associations described are useful for their predictive ability for a number of traits including host resistance. The industry utility of the invention is that young unmeasured progeny can be genotyped and their breeding worth predicted.

### REFERENCES

Abecasis G R and Cookson W O 2000. GOLD—graphical overview of linkage disequilibrium. Bioinformatics 16:182-3
http://www.sph.umich.edu/csg/abecasis/GOLD/index.html
Amer P R 2000. Trait economic weights for genetic improvement with SIL. Proceedings of the New Zealand Society of Animal Production 60:189-191
Beh K J, Hulme D J, Callaghan M J, Leish Z, Lenane I, Windon R G, Maddox J F. 2002. A genome scan for quantitative trait loci affecting resistance to Trichostrongylus colubriformis in sheep. Anim Genet. 33:97-106.
Band, M. R., Larson, J. H., Rebeiz, M., Green, C. A., Heyen, D. W., Donovan, J., Windish, R., Steining, C., Mahyuddin, P., Womack, J. E., et al. 2000. An ordered comparative map of the cattle and human genomes. Genome Res. 10: 1359-1368.
Boutin-Ganache I, Raposo M, Raymond M, Deschepper C F 2001. M13-tailed primers improve the readability and usability of microsatellite analyses performed with two different allelesizing methods. BioTechniques 31:24-28.
Crawford A M, Paterson K A, Dodds K G, Diez Tascon C, Williamson P A, Roberts Thomson M, Bisset S A, Beattie A E, Greer G J, Green R S, Wheeler R, Shaw R J, Knowler K, McEwan J C. Discovery of quantitative trait loci for resistance to parasitic nematode infection in sheep: I. Analysis of outcross pedigrees.
BMC Genomics. 2006 Jul. 18; 7:178.
Dodds, K G and McEwan J C. 1997. Calculating exact probabilities of allele frequency differences in divergent selection lines. Proc. Assoc. Advm. Anim. Breed. Genet. 12:556-560
Ede A J, Pierson C A & Crawford A M 1995. Ovine microsatellites at the OarCP34, OarCP38, OarCP43, OarCP49, OarCP73, OarCP79 and OarCP99 loci. Anim. Genet. 26: 129-31.
Eng S L, Owens E, Womack J E, Cockett N E (2004) Development of an ovine whole-genome radiation hybrid panel. Plant & Animal Genome XII (San Diego, Calif.) P650.
Fan, R, Jung, J and Jin, L (2006) High-resolution association mapping of quantitative trait loci: A population-based approach. Genetics 172: 663-686.
Galloway S M, McNatty K P, Cambridge L M, Laitinen M P, Juengel J L, Jokiranta T S, McLaren R J, Luiro K, Dodds K G, Montgomery G W, Beattie A E, Davis G H, Ritvos O. 2000. Mutations in an oocyte-derived growth factor gene (BMP15) cause increased ovulation rate and infertility in a dosage-sensitive manner. Nat Genet. 25:279-83.
Geenty K G 2000. Sheep industry vision and SIL. Proceedings of the New Zealand Society of Animal Production 60:180-183
Johnson P L, McEwan J C, Dodds K G, Purchas R W, Blair H T. 2005. A directed search in the region of GDF8 for quantitative trait loci affecting carcass traits in Texel sheep. J Anim Sci. 2005 83:1988-2000.
Maddox J F, Davies K P, Crawford A M, Hulme D J, Vaiman D, Cribiu E P, Freking B A, Beh K J, Cockett N E, Kang N, Riffkin C D, Drinkwater R, Moore S S, Dodds K G, Lumsden J M, van Stijn T C, Phua S H, Adelson D L, Burkin H R, Broom J E, Buitkamp J, Cambridge L, Cushwa W T, Gerard E, Galloway S M, Harrison B, Hawken R J, Hiendleder S, Henry H M, Medrano J F, Paterson K A, Schibler L, Stone R T, van Hest B. 2001. An enhanced linkage map of the sheep genome comprising more than 1000 loci. Genome Res. 11:1275-89.
McEwan (2006) attached as Appendix 3.
McLean N J, Jopson N B, Campbell A W, Knowler K, Behrent M, Cruickshank G, Logan C M, Muir P D, Wilson T, McEwan J C 2006. An evaluation of sheep meat genetics in New Zealand: The central progeny test (CPT). Proceedings of the New Zealand Society of Animal Production 66: 368-372
Morris, C A, Wheeler, M, Watson, T G, Hosking, B C & Leathwick, D 2005. Direct and correlated responses to selection for high or low fecal nematode egg count in Perendale sheep. NZ. J. Agr. Res. 48:1-10.
Morris C A, Vlassoff A, Bisset S A, Baker R L, Watson T G, West C J, and Wheeler M. 2000. Continued selection of Romney sheep for resistance or susceptibility to nematode infection: estimates of direct and correlated responses. Anim Sci 70:17-27
Mrode R A, 1996. Linear Models for the Prediction of Animal Breeding Values 187 pp CAB International ISBN 0 85198 996 9
Newman S A, Dodds K G, Clarke J N, Garrick D J, McEwan J C 2000. The Sheep Improvement Limited (SIL) genetic engine. Proceedings of the New Zealand Society of Animal Production 60: 195-197
Newman (2007) attached as appendix 6.
Robinson A J, Love C G, Batley J, Barker G, Edwards D. 2004. Simple sequence repeat marker loci discovery using SSR primer. Bioinformatics. 20:1475-6.
Slatkin and Excoffier 1995. Mol Biol Evol 12:921-7

Saito, D. S., Saitoh, T., Nishium, I. 2005. Isolation and characterization of microsatellite markers in Ijima's leaf warbler, Phylloscopus ijimae (Ayes: Sylviidae). Molecular Ecology Notes 5:666-668
Slonim, D., Kruglyak, L., Stein, L., and Lander, E. 1997. Building human genome maps with radiation hybrids. J. Comput. Biol. 4: 487-504
Young and Walker (2007 a) attached as Appendix 1.
Young and Walker (2007 b) attached as Appendix 2.
Young (2005) attached as Appendix 4.
Young (2006) attached as Appendix 5.

The above Examples illustrate practice of the invention. It will be appreciated by those skilled in the art that numerous variations and modifications may be made without departing from the spirit and scope of the invention.

1. A method for identifying an ovine with a genotype indicative of at least two altered performance traits, the method including the step of detecting, in a sample derived from the ovine, the presence of at least one allele of the CP34 simple sequence repeat (SSR) marker, or at least one allele of a marker in linkage disequilibrium (LD) with CP34, wherein the presence of the allele is indicative of the altered performance traits in the ovine.

2. The method of claim 1 in which the performance trait is selected from the group consisting of: weaning weight (WWT), body weight at 8 months (LW8), body weight at 12 months (LW12), carcass weight (CW), adult ewe weight (EWT), eye muscle width (EMW), eye muscle depth (EMD), eye muscle area (EMA), fat depth (FD), carcass fat weight (FAT), carcass lean muscle weight (LEAN), number of lambs born (NLB), lamb fleece weight (LFW), hogget fleece weight (FW12), ewe (adult) fleece weight (EFW), hogget fibre diameter (FDIAM), and resistance to gastrointestinal parasitic nematode infection.

3. A method for identifying an ovine with a genotype indicative of at least one altered performance traits selected from the group consisting of: weaning weight (WWT), body weight at 8 months (LW8), body weight at 12 months (LW12), carcass weight (CW), adult ewe weight (EWT), eye muscle width (EMW), eye muscle depth (EMD), eye muscle area (EMA), fat depth (FD), carcass fat weight (FAT), carcass lean muscle weight (LEAN), number of lambs born (NLB), lamb fleece weight (LFW), hogget fleece weight (FW12), ewe (adult) fleece weight (EFW), hogget fibre diameter (FDIAM), and resistance to gastrointestinal parasitic nematode infection, the method including the step of detecting, in a sample derived from the ovine, the presence of at least one allele of the CP34 simple sequence repeat (SSR) marker, or at least one allele of a marker in linkage disequilibrium (LD) with CP34, wherein the presence of the allele is indicative of the altered performance traits in the ovine.

4. A method for identifying an ovine with a genotype indicative of at least one altered performance traits selected from the group consisting of: weaning weight (WWT), body weight at 8 months (LW8), body weight at 12 months (LW12), carcass weight (CW), adult ewe weight (EWT), eye muscle width (EMW), eye muscle depth (EMD), eye muscle area (EMA), fat depth (FD), carcass fat weight (FAT), carcass lean muscle weight (LEAN), number of lambs born (NLB), lamb fleece weight (LFW), hogget fleece weight (FW12), ewe (adult) fleece weight (EFW), and hogget fibre diameter (FDIAM), the method including the step of detecting, in a sample derived from the ovine, the presence of at least one allele of the CP34 simple sequence repeat (SSR) marker, or at least one allele of a marker in linkage disequilibrium (LD) with CP34, wherein the presence of the allele is indicative of the altered performance traits in the ovine.

5. The method of claim 1 in which the marker in LD with CP34 is an SSR marker.

6. The method of claim 5 in which the SSR marker in LD with CP34 is selected from the group consisting of BMS1084327, BMS1082942, BMS1082956, BMS1082961, BMS1083945, BMS1083008, BMS1082252, BMS1082669, BMS1082702, BMS1082722, BMS1082831, BMS1887400, BMS1887404, BMS1784528, BMS1600436, BMS1082043, BMS1082045, BMS1081952, BMS1081760, BMS1081860, BMS30480882, BMS30480889, BMS1081770, BMS1081774, RSAD2_{—}1, BMS1081640, BMS1080704, and BMS1080870 as herein defined.

7. The method of claim 1 in which the allele of CP34 is selected from the group consisting of: allele A, allele B, allele C, allele D, allele E, allele F, allele G and allele H, as herein defined.

8. The method of claim 7 in which the allele of CP34 is allele A, G or H.

9. The method of claim 7 in which the allele of CP34 is allele A.

10. The method of claim 7 in which the allele of CP34 is allele C or E.

11. The method of claim 10 in which the allele of CP34 is allele E.

12. The method of claim 1 in which the allele is detected using a polymerase chain reaction (PCR) step.

13. The method of claim 12 in which the allele is detected by amplifying the marker with primers comprising sequence complimentary to sequence of the ovine genome flanking the marker.

14. The method of claim 12 in which the marker is amplified using at least one primer selected from those set forth in Table 2.

15. The method of claim 1 in which the allele is detected by a probe-based methods.

16. The method of claim 15 in which the allele is detected by a probe comprising the sequence of or complementary to the marker.

17. The method of claim 1 in which the presence of a combination of more than one allele of the CP34 SSR marker, or more than one allele of a marker in linkage disequilibrium (LD) with CP34, is detected to identify the ovine.

18. The method of claim 17 in which a combination of at least one allele of the CP34 SSR and at least one allele of a marker in LD with CP34, is detected to identify the ovine.

19. A method for selecting an ovine with at least two altered performance traits, the method comprising selecting an ovine identified by a method of claim 1.

20. A method of selecting an ovine with a genotype indicative of at least one altered performance traits selected from the group consisting of: weaning weight (WWT), body weight at 8 months (LW8), body weight at 12 months (LW12), carcass weight (CW), adult ewe weight (EWT), eye muscle width (EMW), eye muscle depth (EMD), eye muscle area (EMA), fat depth (FD), carcass fat weight (FAT), carcass lean muscle weight (LEAN), number of lambs born (NLB), lamb fleece weight (LFW), hogget fleece weight (FW12), ewe (adult) fleece weight (EFW), hogget fibre diameter (FDIAM), and resistance to gastrointestinal parasitic nematode infection, the method selecting an ovine identified by a method of claim 2.

21. A method of selecting an ovine with a genotype indicative of at least one altered performance traits selected from the group consisting of: weaning weight (WWT), body weight at 8 months (LW8), body weight at 12 months (LW12), carcass weight (CW), adult ewe weight (EWT), eye muscle width (EMW), eye muscle depth (EMD), eye muscle area (EMA), fat depth (FD), carcass fat weight (FAT), carcass lean muscle weight (LEAN), number of lambs born (NLB), lamb fleece weight (LFW), hogget fleece weight (FW12), ewe (adult) fleece weight (EFW), and hogget fibre diameter (FDIAM), the method selecting an ovine identified by a method of claim 3.

22. An isolated polynucleotide comprising an SSR marker selected from the group consisting of BMS1084327, BMS1082942, BMS1082956, BMS1082961, BMS1083945, BMS1083008, BMS1082252, BMS1082669, BMS1082702, BMS1082722, BMS1082831, BMS1887400, BMS1887404, BMS1784528, BMS1600436, BMS1082043, BMS1082045, BMS1081952, BMS1081760, BMS1081860, BMS30480882, BMS30480889, BMS1081770, BMS1081774, RSAD2_{—}1, BMS1081640, BMS1080704, and BMS1080870 as herein defined.

23. A primer suitable for amplifying a polynucleotide of claim 22, the primer comprising a sequence complimentary to sequence of the ovine genome flanking the marker.

24. A primer of claim 23, selected from the primers set forth in Table 2.
